(19) United States
(12) Patent Application Publication
(10) Pub. No.: US 2021/0000855 A1
(43) Pub. Date: Jan. 7, 2021

(54) RETINOL-BINDING PROTEIN 3 (RBP3) AS A PROTECTIVE FACTOR IN NON-DIABETIC RETINAL DEGENERATION

(71) Applicant: Joslin Diabetes Center, Inc., Boston, MA (US)

(72) Inventors: George Liang King, Dover, MA (US); Hillary A. Keenan, Welleley, MA (US)

(21) Appl. No.: 16/866,346

(22) Filed: May 4, 2020

Related U.S. Application Data
(63) Continuation of application No. 15/572,671, filed on Nov. 8, 2017, now abandoned.
(60) Provisional application No. 62/162,439, filed on May 15, 2015.
(60) Provisional application No. 62/161,566, filed on May 14, 2015.

(51) Int. Cl. A61K 31/711; A61K 38/17; A61K 45/06; A61K 35/76; C12N 15/86; A61K 48/00; A61K 31/7088; A61K 38/16; C12N 15/861

(52) U.S. Cl. CPC A61K 31/711; A61K 38/1709; A61K 45/06; A61K 35/76; C12N 15/86; A61K 9/0048; A61K 31/7088; A61K 38/16; C12N 15/861; C12N 2740/16071; C12N 2740/16043; A61K 48/005

(57) ABSTRACT

Methods for increasing retinal thickness in a non-diabetic mammal, comprising administering to the mammal one or both of: (i) a composition comprising RBP3 polypeptide, and/or (ii) a composition comprising a nucleic acid encoding an RBP3 polypeptide.

### CLAIM OF PRIORITY

This application claims the benefit of U.S. patent application Ser. No. 62/161,566, filed on May 14, 2015; and 62/162,439, filed on May 15, 2015. The entire contents of the foregoing are hereby incorporated by reference.

### FEDERALLY SPONSORED RESEARCH OR DEVELOPMENT

This invention was made with Government support under Grant Nos. DK090961-01 and DK094333-01 awarded by the National Institute of Diabetes and Digestive and Kidney Diseases (NIDDK) of the National Institutes of Health. The Government has certain rights in the invention.

### TECHNICAL FIELD

Described are methods for increasing retinal thickness in a non-diabetic mammal, which include administering to the mammal one or both of: (i) a composition comprising RBP3 polypeptide, and/or (ii) a composition comprising a nucleic acid encoding an RBP3 polypeptide.

### BACKGROUND

Retinal thinning resulting from photoreceptor loss is a primary cause of vision loss in retinal degenerative diseases such as Retinitis pigmentosa (RP) (Humayan et al., Invest Ophthalmol Vis Sci. 1999;40:143-148).

### SUMMARY

RBP3, identified as a protective factor in diabetic retinopathy and nephropathy (see, e.g., US2014/0187498), has now been shown to play a role in retinal thickness in non-diabetic eyes. Thus, described herein are methods for increasing retinal thickness in mammals, e.g., non-diabetic mammals, e.g., mammals with or at risk for retinal degeneration associated with retinal thinning, e.g., retinitis pigmentosa, lattice degeneration, or Stargardt disease, or with multiple sclerosis or Gaucher's disease.

Thus, provided herein are methods for increasing retinal thickness in a non-diabetic mammal, the method comprising administering to the mammal one or both of: (i) a composition comprising RBP3 polypeptide, and/or (ii) a composition comprising a nucleic acid encoding an RBP3 polypeptide.

Also provided herein are compositions comprising RBP3 polypeptide, and/or a composition comprising a nucleic acid encoding an RBP3 polypeptide, for use in increasing retinal thickness in a non-diabetic mammal.

In some embodiments, the mammal is a human,

In some embodiments, the mammal has a retinal degenerative disorder associated with retinal thinning.

In some embodiments, the disorder is retinitis pigmentosa, lattice degeneration, or Stargardt disease.

In some embodiments, the disorder is retinal thinning associated with multiple sclerosis or Gaucher's disease.

In some embodiments, the nucleic acid encoding an RBP3 polypeptide is in a viral vector, e.g., an adeno-associated virus or a lentivirus.

In some embodiments, the nucleic acid encoding an RBP3 polypeptide comprises a sequence that is at least 80% identical to nucleotides 1 to 4276, 1 to 3855, 115 to 4276, 115 to 3855, 166 to 3855, 166 to 4276, 1 to 4151, 115 to 4151, or 166 to 4151 of SEQ ID NO:2.

In some embodiments, the RBP3 polypeptide comprises a sequence that is at least 80% identical to amino acids 18-1247 of SEQ ID NO:1.

In some embodiments, the composition is administered by local (ocular) administration.

In some embodiments, the composition is formulated for administration on, in, or into the eye. In some embodiments, the composition is formulated in eye drops, lotions, creams, or ointment.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A. RBP3 expressions by Western blot (WB) in vitreous of mixed population of subjects with type 1 diabetes (Medalists, with 50 or more years of type 1 diabetes, and non-Medalists) and type 2 diabetes separated by grades of diabetic retinopathy (NDM=non-diabetic controls. NPDR=non-proliferative diabetic retinopathy. QPDR=quiescent proliferative diabetic retinopathy. APDR=active proliferative diabetic retinopathy. In box plots, the upper bar, upper box line, middle box line, bottom box line, and bottom bar respectively denote values of 90%, 75%, median, 25% and 10%. N=numbers of eyes (subjects). P-values were tested by Mann-Whitney's U-test for paired comparison.

FIG. 1B. Western blot analysis for RBP3 in human vitreous. Immunoblotting for RBP3 showed specific single bands sized at 135 kDa. Relative intensity of each band was normalized by the average of 3 of non-diabetic controls consistently run in each gel as controls to normalize the variation of experiments (FIG. 1A).

FIGS. 2A-E. Isolation and delipidation of recombinant human RBP3 protein. Human RBP3 protein (hRBP3) was expressed in 293A cells transfected with pCMV-hRBP3-His plasmid, shown in 2A, by FuGene HD (Promega Corp., Madison, Wis.) and purified by the centrifugal filter (Amicon Ultracel 50K). 2B and 2C show the purity and specificity of RBP3 protein was confirmed by Coomassie Blue staining and western blot analysis. To test the effect of retinoid and lipids binding to hRBP3, lipid-free RBP3 (apo-RBP3) was generated by delipidation methods. 2D and 2E show the effects of hRBP3 on Akt and ERK1/2 pathways in bovine retinal pericytes (BRPCs) and bovine retinal endothelial cells (BRECs) were assessed by western blot analysis with the antibodies detecting phosphorylation of Akt/ERK1/2. Inactivated hRBP3 was obtained by boiling (boiled-RBP3). Cells were incubated for 10 min with indicated concentrations of hRBP3s after overnight starvation in DMEM with 0.1% BSA. Ratio of p-Akt and p-ERK1/2 to total Akt and ERK1/2 were quantified by western blot and shown as fold-change to basal condition (0 μM). *P<0.05 and †P<0.01 to basal condition (0 μM).

FIGS. 3A-F. Human RBP3 (hRBP3) effects on phosphorylation of Akt and ERK1/2 in bovine retinal pericytes (BRPCs; 2A) and endothelial cells (BRECs; 2B). Cells were incubated for 10 min with indicated concentrations of hRBP3 after overnight starvation in DMEM with 0.1% BSA. Ratio of p-Akt and p-ERK1/2 to total Akt and ERK1/2 were quantified by western blot and shown as fold-change to basal condition (0 μM). *p<0.05 and †p<0.01 to basal condition (0 μM). 3C shows RBP3 effect on pericyte apoptosis by DNA fragmentation assay. Apoptosis was induced by 72 hrs exposure to high glucose (25 mM) or LPS (500 ng/mL for 1 hr). hRBP3 was added 24 hrs before measurement of DNA fragmentation. 3D shows effects of hRBP3 or Medalist vitreous with high RBP3 expression in protected eye on high glucose- or VEGF-induced endothelial migration by scratch assay in BRECs. 3E shows a pair of immunoblots showing the effects of RBP3 (0.25 μg/ml) and VEGF (2.5 ng/ml) costimulation for 10 minutes on endothelial migration by scratch assay in BRECs. The boxed areas in 3E are reproduced and enlarged at the top of 3F. The bottom of 3F is a graph showing quantification of the fold change in p-Tyr and VEGFR2 under single and costimulatory conditions. Vitreous RBP3 concentration was adjusted. P-values were tested by 2-tailed unpaired t-tests.

FIGS. 4A-E. Establishment of a genetic treatment model with hRBP3 overexpression in the subretina. We generated a lentiviral vector expressing RBP3 driven by CMV-promoter as shown in 4A. 4B indicates the time course of the overall in vivo experimental period. Subretinal injections of the lentivirus at the concentrations of luciferase-GFP (OD: 2.5×10⁶ IFU) and a cocktail of RBP3/luciferase-GFP (OS: 2.25/0.25×10⁶ IFU) were performed by a trans-corneal method at 2 weeks of age to express hRBP3 and luciferase reporter gene. Diabetes was induced by intraperitoneal injection of streptozotocin (STZ; Sigma-Aldrich, Milwaukee, Wis.; 55 mg/kgBW) at 3-4 week intervals. We confirmed luciferase expression in rat eyes at one week, three months and six months after injection using in vivo imaging system (IVIS Lumina system; Caliper Life Sciences, Hopkinton, Mass.) with intraperitoneal injection of D-Luciferin Firefly (50 mg/g BW, Caliper Life Sciences). The results of IVIS detecting luciferase activities are shown in 4C (time-course). 4D shows the expressions of total RBP3 and exogenous RBP3 tagged with FLAG and Myc by western blot analysis. NDM=non-diabetic rats. DM=diabetic rats. 4E shows the correlation of luciferase activity and the expression of RBP3.

FIGS. 5A-E. Protective effects of subretinal overexpression of hRBP3 against neural retina dysfunction and decrease of retinal thickness in STZ induced diabetic Lewis rats. NDM=non-diabetic rats. DM=diabetic rats. RBP3-=eye injected with luciferase gene only. RBP3+=eye injected with hRBP3 and luciferase genes. 5A shows scotopic response of neural retina to light flash indicated as amplitudes of oscillatory potential 1, A-wave and B-wave by dark-adapted electroretinogram (ERG). P-values were tested by 2-tailed unpaired t-tests. 5B and 5D show thicknesses of retina and retinal sub-layers measured by optical coherence tomography (OCT); OCT images are shown in 5C and 5E. In the left panel of 5C, the inner plexiform layer (IPL) is 0.042, the inner nuclear layer (INL) is 0.028, the outer nuclear layer (ONL) is 0.054, the inner segment ellipsoid and end tip (ISE+ET) is 0.04, and the Total is 0.203; in the right panel of 5C, the IPL is 0.043, the INL is 0.025, the ONL is 0.052, the ISE+ET is 0.048, and the Total is 0.216. 5D shows ONL thicknesses in OD: luciferase-GFP and OS: RBP3+luciferase-GFP animals. P-values were tested by ANOVA with Fisher's LSD test. *p<0.05 and **p<0.01 tested by ANOVA. N=6 for each.

FIGS. 6A-C. Effects of subretinal overexpression of hRBP3 in the retina of normal and STZ induced diabetic Lewis rats. NDM=non-diabetic rats. DM=diabetic rats. RBP3-=eye injected with luciferase gene only. RBP3+=eye injected with hRBP3 and luciferase genes. 6A shows quantified acellular capillaries in retinal vascular pathology. 6B shows retinal vascular permeability assessed using the Evans Blue method. 6C shows VEGF expression in vitreous measured by rat VEGF ELISA. P-values were tested by 2-tailed unpaired t-tests.

### DETAILED DESCRIPTION

RBP3 was identified as a potential protective factor against diabetic retinopathy by comparison of individuals with and without proliferative diabetic retinopathy (PDR). The biological roles of human RBP3 were characterized in the retina using retinal vascular cells and eyes of diabetic and normal animals. As demonstrated herein, RBP3 expression can be recovered by genetic therapy, and this treatment increases retinal thickness. RBP3 is thus a therapeutic target for retinal degeneration associated with retinal thinning, e.g., retinitis pigmentosa, lattice degeneration, or Stargardt disease.

### Retinol-Binding Protein 3 (RBP3)

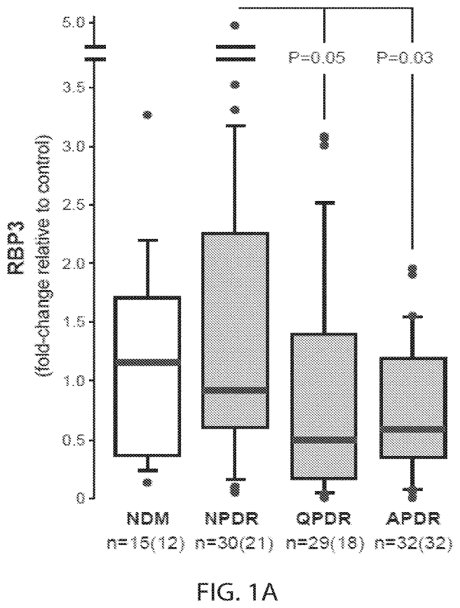

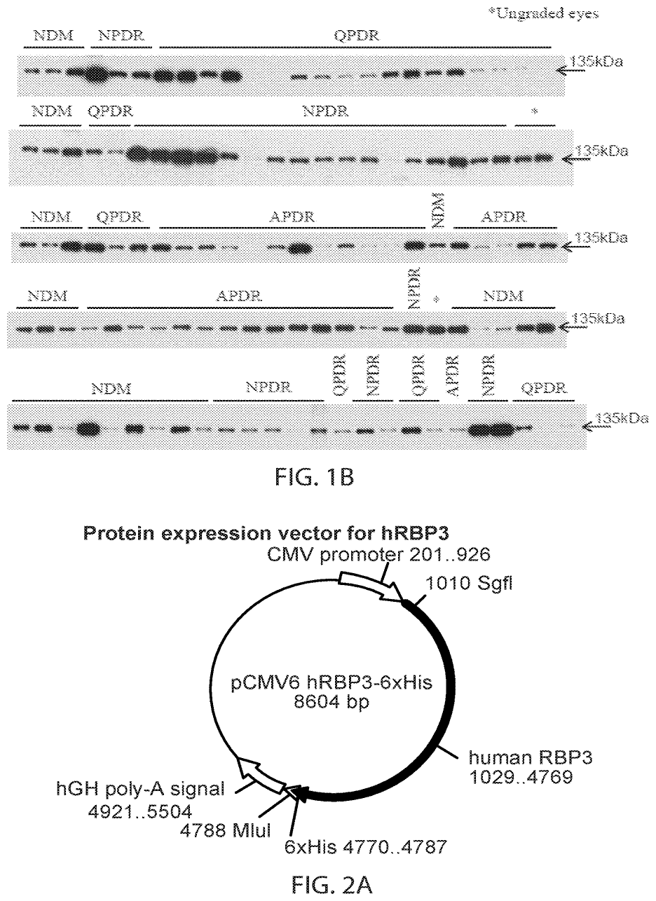

RBP3, a 135 kDa glycolipoprotein highly conserved among mammalian species,²¹ is expressed specifically by rod and cone photoreceptors in the retina and secreted into the interphotoreceptor space.^{22,23} RBP3 is also localized in vitreous humor and aqueous.^{24,25} As shown in FIG. 1, RBP3 expression level in human vitreous has a certain degree of variability. In addition, the present and previous proteomic analyses using human vitreous revealed that RBP3 was one of the proteins paradoxically decreased in PDR.^{15,29,30} RBP3 expression in human retinoblastoma cells was downregulated in a high glucose condition possibly by the stimulation of inflammatory cytokines.²⁶ RBP3 is negatively regulated at the transcriptional level by a blockade of elongation complexes during retinal development.²⁷

An exemplary sequence for human RBP3 can be found in GenBank at Acc. No. NM_002900.2 (nucleic acid) and NP_002891.1 (protein); the protein sequence is reproduced here:

| (SEQ ID NO: 1) | |
|---|---|
| 1 | mmrewvllms vllcglagpt hlfqpslvld makvlldnyc fpenllgmqe aiqqaikshe |
| 61 | ilsisdpqtl asvltagvqs slndprlvis yepstpeppp qvpaltslse eellawlqrg |
| 121 | lrhevlegnv gylrvdsvpg qevlsmmgef lvahvwgnlm gtsalvldlr hctggqvsgi |
| 181 | pyiisylhpg ntilhvdtiy nrpsntttei wtlpqvlger ygadkdvvvl tssqtrgvae |
| 241 | diahilkqmr raivvgertg ggaldlrklr igesdffftv pvsrslgplg ggsqtwegsg |
| 301 | vlpcvgtpae qalekalail tlrsalpgvv hclqevlkdy ytivdrvptl lqhlasmdfs |
| 361 | tvvseedlvt klnaglqaas edprllvrai gptetpswpa pdaaaedspg vapelpedea |
| 421 | irqalvdsvf qvsvlpgnvg ylrfdsfada svlgvlapyv lrqvweplqd tehlimdlrh |
| 481 | npggpssavp lllsyfqgpe agpvhlftty drrtnitqeh fshmelpgpr ystqrgvyll |
| 541 | tshrtataae efaflmqslg wativgeita gnllhtrtvp lldtpegsla ltvpvltfid |
| 601 | nhgeawlggg vvpdaivlae ealdkagevl efhqslgalv egtghlleah yarpevvgqt |
| 661 | sallraklaq gayrtavdle slasqltadl qevsgdhrll vfhspgelvv eeapppppav |
| 721 | pspeeltyli ealfktevlp gqlgylrfda maeletvkav gpqlvrlvwq qlvdtaalvi |
| 781 | dlrynpgsys taipllcsyf feaeprqhly svfdratskv tevwtlpqva gqrygshkdl |
| 841 | yilmshtsgs aaeafahtmq dlqratvige ptaggalsvg iyqvgssply asmptqmams |
| 901 | attgkawdla gvepditvpm sealsiaqdi valrakvptv lqtagklvad nyasaelgak |
| 961 | matklsglqs rysrvtseva laeilgadlq mlsgdphlka ahipenakdr ipgivpmqip |
| 1021 | spevfeelik fsfhtnvled nigylrfdmf gdgelltqvs rllvehiwkk imhtdamiid |
| 1081 | mrfniggpts sipilcsyff degppvlldk iysrpddsys elwthaqvvg erygskksmv |
| 1141 | iltssvtagt aeeftyimkr lgralvigev tsggcqppqt yhvddtnlyl tiptarsvga |
| 1201 | sdgsswegvg vtphvvvpae ealarakeml qhnqlrvkrs pglqdhl |

SEQ ID NO:1 includes the entire sequence; as amino acids 1-17 are predicted to be a signal sequence, in some embodiments, only amino acids 18-1247 of SEQ ID NO:1 are used. See, e.g., Liou et al., Somat. Cell Molec. Genet. 13: 315-323, 1987.

In some embodiments, the sequence of RBP3 used in the present compositions and methods is about 80%, 85%, 90%, 95%, 99% or 100% identical to SEQ ID NO:1.

To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 80% (in some embodiments, about 85%, 90%, 95%, or 100% of the length of the reference sequence) is aligned. The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

### Methods of Treatment

The methods described herein include methods for increasing retinal thickness in a subject, e.g., for the treatment of retinal degenerative disorders associated with retinal thinning. In some embodiments, the disorder is retinitis pigmentosa, lattice degeneration, or Stargardt disease. In some embodiments, the disorder is retinal thinning associated with multiple sclerosis or Gaucher's disease. Generally, the methods include administering a therapeutically effective amount of RBP3 protein or nucleic acid encoding RBP3 protein as described herein, to a subject who is in need of, or who has been determined to be in need of, such treatment. In preferred embodiments, the subject does not have diabetes, and/or does not have a mutation in RBP3, e.g., does not have a D1080N mutation (c.3238G-A transition in exon 2 of the RBP3 gene) described in den Hollander et al., Invest. Ophthal. Vis. Sci. 50: 1864-1872, 2009, or a mutation in RBP3 as described in Ksantini et al., Ophthal. Genet. 31: 200-204, 2010, Li et al., J. Biol. Chem. 288: 11395-11406, 2013, or Liou et al., J. Neurosci. 18: 4511-4520, 1998.

As used in this context, to “treat” means to ameliorate at least one symptom of the disorder associated with retinal thinning. Often, retinal thinning results in loss of visual acuity or risk of retinal tears or detachment; thus, a treatment can result in reduction of the risk of retinal tears or detachment and a return or approach to normal visual acuity. Administration of a therapeutically effective amount of a compound described herein for the treatment of a condition associated with retinal thinning will result in one or more of a decreased rate of retinal thinning, preservation of retinal thickness, and/or an increase in retinal thickness (i.e., an increase in thickness as compared to before administration of the treatment).

### Subjects

The methods described herein can be used to treat subjects who have developed retinal thinning, e.g., a subject who has retinitis pigmentosa, lattice degeneration, Stargardt disease, or retinal thinning associated with multiple sclerosis or Gaucher's disease. Methods for diagnosing these diseases are known in the art; for example, retinal thickness can be determined using optical coherence tomography (OCT) imaging. In some embodiments, the thickness of the outer nuclear layer (ONL) of the retina is measured and considered.

The presence of retinal thinning can be diagnosed based on the presence of a retinal thickness that is below a reference level (of thickness). Suitable reference levels for retinal thickness can be determined based on epidemiological studies, e.g., using specific OCT imaging devices, e.g., as described in Chan et al., Arch Ophthalmol. 2006 February; 124(2): 193-198, and can depend on the age of the subject, see, e.g., Alamouti and Funk, Br J Ophthalmol. 2003 July; 87(7): 899-901.

In some embodiments, the methods described herein include detecting the presence of reduced levels of RBP3 protein or mRNA in the mammal, e.g., in the eye of the mammal, and optionally selecting a subject who has levels of RBP3 protein or mRNA below a reference level (e.g., a reference level that represents a level of RBP3 in a normal subject) for treatment using a method described herein. In some embodiments, the methods described herein include detecting the presence of reduced levels of RBP3 protein or mRNA in a mammal who does not have retinal thinning, e.g., in the eye of the mammal, and determining that the subject is at risk (i.e., has a higher risk that the general population) of developing retinal thinning when the subject has levels of RBP3 protein or mRNA below a reference level (e.g., a reference level that represents a level of RBP3 in a subject with a normal level of risk of developing retinal thinning).

The methods include obtaining a sample from a subject, and evaluating the presence and/or level of RBP3 in the sample, and comparing the presence and/or level with one or more references, e.g., a control reference that represents a normal level of RBP3, e.g., a level in an unaffected subject, and/or a disease reference that represents a level of the proteins associated with retinal thinning, e.g., a level in a subject having retinal thinning.

As used herein the term “sample”, when referring to the material to be tested for the presence of a biological marker using the method of the invention, includes inter alia vitreous, whole blood, plasma, or serum. The type of sample used may vary depending upon the identity of the biological marker to be tested and the clinical situation in which the method is used. Various methods are well known within the art for the identification and/or isolation and/or purification of a biological marker from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The presence and/or level of a protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance.

For example, levels of RBP3 protein in a sample (e.g., a sample of vitreous, blood, serum, or plasma) can be detected using an RBP3-binding antibody or fragment thereof. The term “antibody” as used herein refers to an immunoglobulin molecule or an antigen-binding portion thereof. Examples of antigen-binding portions of immunoglobulin molecules include F(ab) and F(ab′)₂ fragments, which retain the ability to bind antigen. The antibody can be polyclonal, monoclonal, recombinant, chimeric, de-immunized or humanized, fully human, non-human, (e.g., murine), or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. Antibodies that bind to RBP3 are known in the art and commercially available, e.g., from Abbexa, Abcam, Abbiotec, Abnova, Atlas Antibodies, Life Technologies, OriGene, Novus Biologicals, United States Biological, and Santa Cruz Biotechnolgy, Inc.; additional antibodies and fragments thereof can be generated using methods known in the art, see, e.g., Harlow et. al., editors, Antibodies: A Laboratory Manual (1988); Goding, Monoclonal Antibodies: Principles and Practice, (N.Y. Academic Press 1983); Howard and Kaser, Making and Using Antibodies: A Practical Handbook (CRC Press; 1st edition, Dec. 13, 2006); Kontermann and Dübel, Antibody Engineering Volume 1 (Springer Protocols) (Springer; 2nd ed., May 21, 2010); Lo, Antibody Engineering: Methods and Protocols (Methods in Molecular Biology) (Humana Press; Nov. 10, 2010); and Dübel, Handbook of Therapeutic Antibodies: Technologies, Emerging Developments and Approved Therapeutics, (Wiley-VCH; 1 edition Sep. 7, 2010).

The antibody can be coupled to a detectable or imaging agent. Such agents are well known in the art and include paramagnetic agents, bioluminescent or fluorescent labels (e.g., GFP, FITC, rhodamine, or Texas Red), radioactive isotopes, and colorimetric/enzymatic agents (e.g., HRP, B-galactosidase). In a preferred embodiment, the antibody is coupled to a paramagnetic agent, e.g., a paramagnetic nanoparticle, e.g., cross-linked iron oxide (CLIO) nanoparticles; see, e.g., US 20110046004; Josephson et al., Bioconjug. Chem., 10(2):186-91 (1999).

In some embodiments, an ELISA method may be used, e.g., wherein the wells of a mictrotiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

In some embodiments, an IHC method may be used. IHC provides a method of detecting a biological marker in situ. The presence and exact cellular location of the biological marker can be detected. Typically a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

Mass spectrometry, and particularly matrix-assisted laser desorption/ionization mass spectrometry (MALDI-MS) and surface-enhanced laser desorption/ionization mass spectrometry (SELDI-MS), is useful for the detection of biomarkers of this invention. (See U.S. Pat. Nos. 5,118,937; 5,045,694; 5,719,060; and 6,225,047)

The presence and/or level of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559) ; RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999,W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of RBP3. Measurement of the level of RBP3 can be direct or indirect. For example, the abundance levels of RBP3 can be directly quantitated. Alternatively, the amount of a biomarker can be determined indirectly by measuring abundance levels of RBP3 cDNA, amplified RNAs or DNAs, or by measuring quantities or activities of RNAs, or other molecules that are indicative of the expression level of the biomarker. In some embodiments a technique suitable for the detection of alterations in the structure or sequence of nucleic acids, such as the presence of deletions, amplifications, or substitutions, can be used for the detection of biomarkers of this invention.

RT-PCR can be used to determine the expression profiles of RBP3 (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes, such as glyceraldehyde-3-phosphate dehydrogenase (GAPDH), beta-actin (ACTB), lactate dehydrogenase A (LDHA), ribosomal protein L5 (RPL5), ubiquitin C (UBC), peptidylprolyl isomerase A (PPIA), TATA-box binding protein (TBP1), and/or hypoxanthine guanine phosphoribosyl transferase (HPRT1), are most commonly used.

Gene arrays are prepared by selecting probes which comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

In some embodiments, the presence and/or level of RBP3 is comparable to the presence and/or level of the protein(s) in the disease reference, and the subject has no overt signs or symptoms of retinal thinning, then the subject has an increased risk of developing retinal thinning, and a treatment, e.g., as known in the art or as described herein, can be administered to reduce the risk of developing retinal thinning. In some embodiments, once it has been determined that a person has retinal thinning, or has an increased risk of developing retinal thinning, then a treatment, e.g., as known in the art or as described herein, can be administered.

Suitable reference values can be determined using methods known in the art, e.g., using standard clinical trial methodology and statistical analysis. The reference values can have any relevant form. In some cases, the reference comprises a predetermined value for a meaningful level of RBP3, e.g., a control reference level that represents a normal level of RBP3, e.g., a level in an unaffected subject (with a normal retinal thickness) or a subject who is not at risk of developing retinal thinning, and/or a disease reference that represents a level of RBP3 associated with retinal thinning.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have a disorder described herein (e.g. retinal thinning). In the present methods, the subjects do not have diabetes.

A disease reference subject is one who has (or has an increased risk of developing) retinal thinning. An increased risk is defined as a risk above the risk of subjects in the general population.

Thus, in some cases the level of RBP3 in a subject being less than or equal to a reference level of RBP3 is indicative of a clinical status (e.g., indicative of a disorder as described herein, e.g., retinal thinning. In other cases the level of RBP3 in a subject being greater than or equal to the reference level of RBP3 is indicative of the absence of disease or normal risk of the disease (i.e., the same risk as the general population). In some embodiments, the amount by which the level in the subject is less than the reference level is sufficient to distinguish a subject from a control subject, and optionally is statistically significantly less than the level in a control subject. In cases where the level of RBP3 in a subject being equal to the reference level of RBP3, the “being equal” refers to being approximately equal (e.g., not statistically different).

The predetermined value can depend upon the particular population of subjects (e.g., human subjects) selected. For example, an apparently healthy population will have a different ‘normal’ range of levels of RBP3 than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

### Gene Therapy

Described herein are methods in which a nucleic acid encoding an RBP3 polypeptide or active fragment thereof, e.g., incorporated into a gene transfer construct , is used to increase retinal thickness, e.g., to treat a condition associated with retinal thinning as described herein. In some embodiments, the nucleotide sequence encoding human RBP3 as described herein is at least about 75% identical to the reference sequence of human RBP3 found in GenBank at Acc. No. NM 002900.2 (nucleic acid), e.g., SEQ ID NO:2. In some embodiments, the nucleotide sequences are about 80%, 85%, 90%, 95%, 99% or 100% identical to a sequence encoding a mature or full length human RBP3, e.g., a sequence comprising nucleotides 1 to 4276, 1 to 3855, 115 to 4276, 115 to 3855, 166 to 3855, 166 to 4276, 1 to 4151, 115 to 4151, or 166 to 4151 of SEQ ID NO:2 (nucleotides 115 to 165 encode a signal sequence).

Exemplary Human RBP3 sequence:

| (SEQ ID NO: 2) | |
|---|---|
| 1 | tgtccaccag ctgagaagga caagggcgga aggcagctgc acagagcagg gccacggcct |
| 61 | tgcacacagt ccagggagct tttgtgcagg agccaggcct ccccctgggt ccccatgatg |
| 121 | agagaatggg ttctgctcat gtccgtgctg ctctgtggcc tggctggccc cacacacctg |
| 181 | ttccagccaa gcctggtgct ggacatggcc aaggtcctct tggataacta ctgcttcccg |
| 241 | gagaacctgc tgggcatgca ggaagccatc cagcaggcca tcaagagcca tgagattctg |
| 301 | agcatctcag acccgcagac gctggccagt gtgctgacag ccggggtgca gagctccctg |
| 361 | aacgatcctc gcctggtcat ctcctatgag cccagcaccc ccgagcctcc cccacaagtc |
| 421 | ccagcactca ccagcctctc agaagaggaa ctgcttgcct ggctgcaaag gggcctccgc |
| 481 | catgaggttc tggagggtaa tgtgggctac ctgcgggtgg acagcgtccc gggccaggag |
| 541 | gtgctgagca tgatggggga gttcctggtg gcccacgtgt gggggaatct catgggcacc |
| 601 | tccgccttag tgctggatct ccggcactgc acaggaggcc aggtctctgg cattccctac |
| 661 | atcatctcct acctgcaccc agggaacacc atcctgcacg tggacactat ctacaaccgc |
| 721 | ccctccaaca ccaccacgga gatctggacc ttgccccagg tcctgggaga aaggtacggt |
| 781 | gccgacaagg atgtggtggt cctcaccagc agccagacca ggggcgtggc cgaggacatc |
| 841 | gcgcacatcc ttaagcagat gcgcagggcc atcgtggtgg gcgagcggac tgggggaggg |
| 901 | gccctggacc tccggaagct gaggataggc gagtctgact tcttcttcac ggtgcccgtg |
| 961 | tccaggtccc tggggcccct tggtggaggc agccagacgt gggagggcag cggggtgctg |
| 1021 | ccctgtgtgg ggactccggc cgagcaggcc ctggagaaag ccctggccat cctcactctg |
| 1081 | cgcagcgccc ttccaggggt agtccactgc ctccaggagg tcctgaagga ctactacacg |
| 1141 | ctggtggacc gtgtgcccac cctgctgcag cacttggcca gcatggactt ctccacggtg |
| 1201 | gtctccgagg aagatctggt caccaagctc aatgccggcc tgcaggctgc gtctgaggat |
| 1261 | cccaggctcc tggtgcgagc catcgggccc acagaaactc cttcttggcc cgcgcccgac |
| 1321 | gctgcagccg aagactcacc aggggtggcc ccagagttgc ctgaggacga ggctatccgg |
| 1381 | caagcactgg tggactctgt gttccaggtg tcggtgctgc caggcaatgt gggctacctg |
| 1441 | cgcttcgata gttttgctga cgcctccgtc ctgggtgtgt tggccccata tgtcctgcgc |
| 1501 | caggtgtggg agccgctaca ggacacggag cacctcatca tggacctgcg ccacaaccct |
| 1561 | ggagggccat cctctgctgt gcccctgctc ctgtcctact tccagggccc tgaggccggc |
| 1621 | cccgtgcacc tcttcaccac ctatgatcgc cgcaccaaca tcacgcagga gcacttcagc |
| 1681 | cacatggagc tcccgggccc acgctacagc acccaacgtg gggtgtatct gctcaccagc |
| 1741 | caccgcaccg ccacggccgc ggaggagttc gccttcctta tgcagtcgct gggctgggcc |
| 1801 | acactggtag gtgagatcac cgcgggcaac ctgctgcaca cccgcacggt gccgctgctg |
| 1861 | gacacacccg aaggcagcct cgcgctcacc gtgccggtcc tcaccttcat cgacaatcac |
| 1921 | ggcgaggcct ggctgggtgg tggagtggtg cccgatgcca tcgtgctggc cgaggaggcc |
| 1981 | ctggacaaag cccaggaagt gctggagttc caccaaagcc tgggggcctt ggtggagggc |
| 2041 | acagggcacc tgctggaggc ccactatgct cggccagagg tcgtggggca gaccagtgcc |
| 2101 | ctcctgcggg ccaagctggc ccagggcgcc taccgcacag ctgtggactt ggagtctctg |
| 2161 | gcctctcagc tcacagcaga cctccaggag gtgtctgggg accaccgctt gctagtgttc |
| 2221 | cacagccctg gcgagctggt ggtagaggaa gcacccccac caccccctgc tgtcccctct |
| 2281 | ccagaggagc tcacctacct tattgaggcc ctgttcaaga cagaggtgct gcccggccag |
| 2341 | ctgggctacc tgcgttttga cgccatggct gaactggaga cagtgaaggc cgtggggcca |
| 2401 | cagctggtgc ggctggtatg gcaacagctg gtggacacgg ctgcgctggt gatcgacctg |
| 2461 | cgctacaacc ctggcagcta ctccacggcc atcccgctgc tctgctccta cttctttgag |
| 2521 | gcagagcccc gccagcacct gtattctgtc tttgacaggg ccacctcaaa agtcacggag |
| 2581 | gtgtggacct tgccccaggt cgccggccag cgctacggct cacacaagga cctctacatc |
| 2641 | ctgatgagcc acaccagtgg ctctgcggcc gaggcctttg cacacaccat gcaggacctg |
| 2701 | cagcgggcca cggtcattgg ggagcccacg gccggaggcg cactctctgt gggcatctac |
| 2761 | caggtgggca gcagcccctt atatgcatcc atgcccaccc agatggccat gagtgccacc |
| 2821 | acaggcaagg cctgggacct ggctggtgtg gagcccgaca tcactgtgcc catgagcgaa |
| 2881 | gccctttcca tagcccagga catagtggct ctgcgtgcca aggtgcccac ggtgctgcag |
| 2941 | acggccggga agctggtggc tgataactat gcctctgccg agctgggggc caagatggcc |
| 3001 | accaaactga gcggtctgca gagccgctac tccagggtga cctcagaagt ggccctagcc |
| 3061 | gagatcctgg gggctgacct gcagatgctc tccggagacc cacacctgaa ggcagcccat |
| 3121 | atccctgaga atgccaagga ccgcattcct ggaattgtgc ccatgcagat cccttcccct |
| 3181 | gaagtatttg aagagctgat caagttttcc ttccacacta acgtgcttga ggacaacatt |
| 3241 | ggctacttga ggtttgacat gtttggggac ggtgagctgc tcacccaggt ctccaggctg |
| 3301 | ctggtggagc acatctggaa gaagatcatg cacacggatg ccatgatcat cgacatgagg |
| 3361 | ttcaacatcg gtggccccac atcctccatt cccatcttgt gctcctactt ctttgatgaa |
| 3421 | ggccctccag ttctgctgga caagatctac agccggcctg atgactctgt cagtgaactc |
| 3481 | tggacacacg cccaggttgt aggtgaacgc tatggctcca agaagagcat ggtcattctg |
| 3541 | accagcagtg tgacggccgg caccgcggag gagttcacct atatcatgaa gaggctgggc |
| 3601 | cgggccctgg tcattgggga ggtgaccagt gggggctgcc agccaccaca gacctaccac |

| | |
|---|---|
| 3661 | gtggatgaca ccaacctcta cctcactatc cccacggccc gttctgtggg ggcctcggat |
| 3721 | ggcagctcct gggaaggggt gggggtgaca ccccatgtgg ttgtccctgc agaagaggct |
| 3781 | ctcgccaggg ccaaggagat gctccagcac aaccagctga gggtgaagcg gagcccaggc |
| 3841 | ctgcaggacc acctgtaggg aagggcccca taggcagagc cccagggcag acagaacctc |
| 3901 | tgggacacac accaagggca ctcctgcagg tggcccggcc tgaggttccc aggagcagca |
| 3961 | aaggggcctg ctgagctctg gttaggttac agctggaggt gtgtatatat acacacacac |
| 4021 | acatgtatat acacatatat atgtgtatgt atatatatgt atatatatat ggctttccaa |
| 4081 | taaccaccta aattttaaca aaggttcctt ctaagtggta gaacttgggg tggtattttt |
| 4141 | accttccttc ttcatacttt gctctttttc ttaaatactc attaatgtgc atatatcatt |
| 4201 | attttcagat gcagctatca ttattccaaa atacaaaata aagaagataa aataaattat |
| 4261 | atacccgagc cattaaaaaa aaaaaaaaa |

These methods can include the use of targeted expression vectors for in vivo transfection and expression of a polynucleotide that encodes an RBP3 polypeptide or active fragment thereof, preferably in particular cell types, especially cells of the retina, e.g., of the ONL of the retina. Expression constructs of such components can be administered in any effective carrier, e.g., any formulation or composition capable of effectively delivering the component gene to cells in vivo. Approaches include insertion of the RBP3 gene into gene transfer and expression constructs such as viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO₄ precipitation carried out in vivo.

Retrovirus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al.

(1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro and Immuno1.158:97-129 (1992)). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). In preferred embodiments, the viral delivery vector is a recombinant AAV2/8 virus.

In addition to viral transfer methods, such as those illustrated above, non-viral methods can also be employed to cause expression of a nucleic acid compound described herein (e.g., a RBP3 nucleic acid) in the tissue of a subject. Typically non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther. 7(22):1896-905 (2000); or Tam et al., Gene Ther. 7(21):1867-74 (2000).

In some embodiments, a nucleic acid encoding RBP3 is entrapped in liposomes bearing positive charges on their surface (e.g., lipofectins), which can be tagged with antibodies against cell surface antigens of the target tissue (Mizuno et al., No Shinkei Geka 20:547-551 (1992); PCT publication WO91/06309; Japanese patent application 1047381; and European patent publication EP-A-43075).

In some embodiments, the nucleic acid (e.g., cDNA) encoding RBP3 is operably linked to regulatory sequences. The term “regulatory sequence” includes promoters, enhancers and other expression control elements (e.g., polyadenylation signals). Regulatory sequences include, e.g., a promoter to drive expression of the RBP3 sequence in a cell, e.g., constitutive expression of a nucleotide sequence, and/or tissue-specific regulatory and/or inducible sequences. Exemplary promoters for use in the present methods include those that drive expression in the eye, e.g., in the retina, e.g., in the ONL, e.g., a rhodopsin (Rho) promoter or rhodopsin kinase (RK) promoter; a chimeric promoter consisting of an enhancer element of interphotoreceptor retinoid-binding protein promoter and a minimal sequence of the human transducin alpha-subunit promoter (IRBPe/GNAT2) (Dyka et al., Adv Exp Med Biol. 2014;801:695-701); synthetic transducin alpha-subunit promoter (synGNAT2/GNAT2) (Id.); an RPE65 promoter (Bainbridge et al., N Engl J Med 2008; 358: 2231-2239); phosphoglycerate kinase 1 (PGK) promoter; elongation factor-1 (EFS) promoter (KOstic et al., Gene Therapy (2003) 10, 818-821); and others.

In clinical settings, the gene delivery systems for the therapeutic gene can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., PNAS USA 91: 3054-3057 (1994)). In preferred embodiments, the gene is delivered by intravitreal injection.

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

### Pharmaceutical Compositions and Methods of Administration

The methods described herein can include the use of pharmaceutical compositions comprising RBP3 proteins and/or nucleic acids as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include systemic (e.g., parenteral and oral) and local (ocular) administration. Thus the methods described herein can include administration of RBP3 proteins in a formulation for administration on, in, or into the eye, e.g., in eye drops, lotions, creams, e.g., comprising microcapsules, microemulsions, nanoparticles, etc. Methods of formulating suitable pharmaceutical compositions for ocular delivery are known in the art, see, e.g., Losa et al., Pharmaceutical Research 10:1 (80-87 (1993); Gasco et al., J. Pharma Biomed Anal., 7(4):433-439 (1989); Fischer et al., Eur J Ophthalmol. 21 Suppl 6:S20-6 (2011); and Tangri and Khurana, Intl J Res Pharma Biomed Sci., 2(4):1541-1442 (2011).

For administration of proteins, ocular administration, e.g., via eye drops, ocular gel, lotion, ointment, or cream; or intraocular, e.g., intravitreal or periocular injection, is preferred. Subconjunctival, sub-Tenon's and juxtamacular injections can also be used.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Bioadhesive polymers most common to ophthalmic drug development include hydroxypropyl methylcellulose (HPMC), carboxymethylcellulose (CMC) and polyacrylic acid (PAA) derivatives, as well as hyaluronic acid (HA). Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811; US 20090136445; Gupta et al., J Pharm Bioallied Sci. 2015 January-March; 7(1): 9-14. Soluble and insoluble ocular inserts, e.g., sustained-release implants, can also be used, e.g., rod-shaped, soluble hydroxy propyl cellulose ocular inserts such as the Lacrisert (Aton Pharma) or Ocusert (Alza Pharmaceuticals). Injectable nanoparticles and microparticles can also be used, e.g., polyion complex (PIC) micelles that are laser-activated. See, e.g., Saettone and Salminen, Adv Drug Deliv Rev 1995;16:95-106; Johnston TP, Dias CS, Mitra AK. Mucoadhesive polymers in ophthalmic drug delivery. In: Mitra AK, ed. Ophthalmic Drug Delivery Systems, v. 130. New York: Marcel Dekker, 2003; Chun DK, Shapiro A, Abelson MB. Ocular Pharmacokinetics. In: Albert DM,

Jakobiec FA, eds. Principles and Practice of Ophthalmology, 3rd ed. Canada: Elsevier, 2008; v. 1; Csernus VJ, Szende B, Schally AV. Release of peptides from sustained delivery systems (microcapsules and microparticles) in vivo. A histological and immunohistochemical study. Int J Pept Protein Res 1990;35:6:557-65; and Masuda I, Matsuo T, Yasuda T, Matsuo N. Gene transfer with liposomes to the intraocular tissues by different routes of administration. Invest Ophthalmol Vis Sci 1996;37:9:1914-20; Gupta et al., J Pharm Bioallied Sci. 2015 January-March; 7(1): 9-14. Single-use, non-preserved unit administration can also be used.

The pharmaceutical compositions can be included in a container, pack, or dispenser together with instructions for administration.

### EXAMPLES

The invention is further described in the following examples, which do not limit the scope of the invention described in the claims.

### Methods

The following materials and methods were used in the Examples herein.

### Study design and population

We conducted this study using data and eye samples from Joslin 50-Year Medalist Study, the Beetham Eye Institute, the California Retina Consultants and the National Disease Research Interchange (NDRI). All study participants provided written informed consent. The institutional review boards at the Joslin Diabetes Center and each participating site approved the study protocols. The study design and the overall characteristics of the Medalists at baseline have been described previously.^{17,18} All subjects were evaluated at the Joslin clinic with medical history, clinical and ophthalmic exam, and blood and urine collection. Methods used for the LS/MS-based proteomic analysis have been reported previously.¹⁵ Retina and vitreous of Medalists with quiescent PDR (QPDR; n=11 eyes, 6 people) no-mild DR (n=6 eyes, 4 people) were analyzed using the criteria of a minimum 1.5-fold increase of peptide numbers with p-value<0.05 in both retina and vitreous by Mann-Whitney U test. Western blot analysis in was performed using human vitreous from a group including non-Medalists with type 1 and 2 diabetes of 83 individuals that consists of non-diabetic controls (NDM, n=12), non-PDR (NPDR, n=21), QPDR (n=18) and active PDR (APDR, n=32).

### Cell-based assays using human RBP3 protein

Human RBP3 protein (hRBP3) was expressed in 293A cells transfected with pCMV-hRBP3-His plasmid by FuGene HD (Promega Corp., Madison, Wis.) and purified by the centrifugal filter (Amicon Ultracel 50K). The purity and specificity of RBP3 protein was confirmed by Coomassie Blue staining and western blot analysis. Lipid-free RBP3 (apo-RBP3) was generated by delipidation methods. Inactivated hRBP3 (apo-RBP3) was obtained by boiling. We isolated primary cultures of bovine retinal pericytes (BRPCs) and bovine retinal endothelial cells (BRECs) by homogenization and a series of filtration steps as reported previously.^{5,19} The effects of hRBP3 on Akt and ERK1/2 pathways in BRPCs and BRECs were assessed by western blot analysis with the antibodies detecting phosphorylation of Akt/ERK1/2 using the following antibodies from Cell Signaling Technology (Danvers, Mass.): P-Akt (#4060), Akt (#9272), P-ERK1/2 (#9101) and ERK1/2 (#4695). DNA fragmentation in BRPCs was measured by quantitation of cytosolic oligonucleosome-bound DNA using ELISA (Roche Molecular Biochemicals) according to the manufacturer's instructions. Scratch assay was performed to assess the effect of hRBP3 on BRECs migration. The percentage of migration area was calculated as the ratio of covered area (original wound area - open wound area) to the original wound area.

### In Vivo Gene Therapy with Human RBP3

Male pups born from Lewis rats (Charles River Laboratories International, Inc., Wilmington, Mass.) were used to develop the model of subretinal overexpression of hRBP3. First, we generated lentiviral vectors expressing RBP3 or luciferase-GFP driven by CMV-promoter (Dr. S. Kissler at Joslin Diabetes Center). We confirmed co-expressed luciferase activity in rat eyes using in vivo imaging system (IVIS Lumina system; Caliper

Life Sciences, Hopkinton, Mass.) with intraperitoneal injection of D-Luciferin Firefly (50 mg/g BW, Caliper Life Sciences), which correlates well with the expression of RBP3. Diabetes was induced by intraperitoneal injection of streptozotocin (STZ; Sigma-Aldrich, Milwaukee, Wis.; 55 mg/kgBW) at 6 week of age and ascertained by blood glucose at fasting>250 mg/dl and fed<450mg/dl. The experimental protocols were approved by the Joslin Diabetes Center Institutional Animal Care and Use Committee. The experiments were carried out in strict accordance with the recommendations in the Guide for the Care and Use of Laboratory Animals of the National Institutes of Health. In vivo scanning of the retina and scotopic elecroretinogram (ERG) were performed at 2 months after STZ injection by using the Bioptigen 840S SD-OCT imaging system (Charlotte, N.C.) and the ERG recording system (PowerLab, ADlnstruments Santa Clara, Calif.) controlled by the LED-driver (WLS-20, Mayo Co., Nagoya, Japan). The Evans blue-dye permeation technique was performed at 2 months after STZ injection to quantify retinal vascular permeability indicated as the rate of plasma extravasation per unit weight of retinal tissue. We assessed retinal vascular pathology at 6 months after STZ injection by identification of acellular capillaries indicated as per square millimeter of retinal area as described below.

### Western Blot Analysis for RBP3 in Human Vitreous

Western blot analysis of the vitreous from the study population including non-Medalists with type 1 and 2 diabetes, as well as NDM (non-diabetes mellitus) controls was performed. 10 ul of each vitreous sample was sonicated for 5 seconds, and boiled in 2×laemmli sample buffer. 5 ul of the resulting sample was loaded onto a 4-20% TBS gel (Biorad). Gels were transferred to nitrocellulose membrane and immunoblotted with 1:2000 diluted rabbit polyclonal anti-RBP3 (abcam) antibody. The blots were scanned and the bands quantified using ImageJ software (NIH), and relative intensity of each band was normalized by the average of the three non-diabetic controls consistently run in each gel to normalize the variation of experiments.

### Isolation and delipidation of recombinant human RBP3 protein

To express human RBP3 protein (hRBP3), 293A cells were split and plated on ten 10cm dishes at the concentration of 3×10⁵ cells/mL. 24 hours later, pCMV-hRBP3-His (2.7 μg) and pAdvantage (0.3 μg) plasmids for each plate were transfected by FuGene HD-based method according to the instruction of product (DNA:FuGene=1:3; Promega Corp., Madison, Wis.). The secreted RBP3 protein in the media were collected and partially purified by the filtration with a centrifugal filter (Amicon Ultracel 50K). The purity and specificity of RBP3 protein was confirmed by Coomassie Blue staining and western blot analysis. Lipid-free RBP3 (apo-RBP3) was generated by delipidation methods as reported previously (Norseen et al., Molecular and cellular biology 2012;32:2010-9). Briefly, lipid-bound hRBP3 (holo-RBP3) was first incubated with 40% butanol-60% diisopropyl ether (DIPE) at 30° C. overnight to remove lipids and retinol in a glass tube. The tube was centrifuged at 5,000 rpm for 5 min, and the bottom phase containing hRBP3 was collected. This step was repeated twice more with 1 hr incubations of 40% butanol-60% (DIPE). Inactivated hRBP3 (boiled-RBP3) was obtained by boiling at 95° C. for 30 min and three times of sonication for 15 sec.

### Cell culture

Fresh calf eyes were obtained from a local slaughterhouse. We isolated primary cultures of bovine retinal pericytes (BRPCs) and bovine retinal endothelial cells (BRECs) by homogenization and a series of filtration steps (143 and 55 μm; top of the 55 μm filter was saved as retinal microvascular cells) as reported previously (Geraldes et al., Nature medicine 2009;15:1298-306; Nayak et al., The Journal of experimental medicine 1988;167:1003-15). BRPCs were subsequently propagated in DMEM with 20% FBS on collagen I-coated dishes (BD Biosciences). BRECs were grown in DMEM with 10% horse serum, 100 μg/m1 heparin and 50 μg/m1 endothelial cell growth supplement (Roche Applied Science) on 2% gelatin-coated dishes. We used cells from passages 3 through 6. We used serum free DMEM with 0.1% BSA for overnight starvation. We exposed cells to 5.6 mM glucose (low glucose) or 25 mM glucose (high glucose) for 72 hours. We adjusted the osmotic pressure in low glucose conditions by adding 19.4 mM mannitol.

### Cell-Based Assays

The effects of hRBP3 on Akt and ERK1/2 pathways in BRPCs and BRECs were assessed by western blot analysis with the antibodies detecting phosphorylation of Akt/ERK1/2. BRPCs and BRECs were plated on 6 well plates with collagen-I and gelatin coating, respectively at the concentration of 5×10⁴ cells per well and grown in growth medium for 24 hours. After starvation overnight in 1 ml of DMEM/0.1% BSA, cells were treated with holo-, apo- and boiled-RBP3 at the indicated concentrations and incubated at 37° C. for 10 min. Then, cells were washed with ice-cold PBS and lysed immediately with RIPA buffer including protease inhibitor. Cell lysates were denatured with ×6 sample loading buffer for immunoblotting with the following antibodies from Cell Signaling: P-Akt (#4060), Akt (#9272), P-ERK1/2 (#9101) and ERK1/2 (#4695).

DNA fragmentation in BRPCs was measured by quantitation of cytosolic oligonucleosome-bound DNA using ELISA (Roche Molecular Biochemicals) according to the manufacturer's instructions. Briefly, cells were grown in 12 well plates at a density of 5×10⁴ cells per well in 1 ml DMEM and 20% FBS. BRPCs were exposed to 5.6 mM or 25 mM glucose condition or LPS (500 ng/mL) in DMEM/0.5% FBS media for 72 hours in presence or absence of rhRBP3 (10 μg/m1), and then lysed directly on the plate. The cytosolic fraction was used as an antigen source in a sandwich ELISA with primary anti-histone antibody coated to the microtiter plate and a secondary anti-DNA antibody coupled to peroxidase. Absorbance values were measured at 405 nm wavelength and with a reference wavelength at 492 nm.

A scratch assay was performed to assess the effect of hRBP3 on BRECs migration as described previously (Liang et al., Nature protocols 2007;2:329-33). Cells were grown to confluence on 0.2% gelatin-coated 35 mm plates in growth media. Scratch wounds were created in confluent monolayers using a sterile p200 pipette tip. Perpendicular marks were placed at intervals of 1 mm across each scratch on the external surface of the well. After the suspended cells were washed, the wounded monolayers were incubated in each conditions of test medium. Every 6 hours up to 24 hours, repopulation of the wounded areas was observed under phase-contrast microscopy (Olympus, Japan). Using the NIH ImageJ image processing program, the size of the denuded area was determined at each time point from digital images. The percentage of migration area was calculated as the ratio of covered area (original wound area−open wound area) to the original wound area.

### In Vivo Study

Pregnant female Lewis rats were obtained from Charles River Laboratories International, Inc. (Wilmington, Mass.). Born male pups were used to develop the model of subretinal overexpression of RBP3. First, we generated lentiviral vectors expressing RBP3 or luciferase-GFP driven by CMV-promoter as previously reported (Kissler et al., Methods in molecular biology 2009;555:109-18). Subretinal injections of lentivirus at the concentrations of luciferase-GFP (OD: 2.5×10⁶ IFU) and a cocktail of RBP3/luciferase-GFP (OS: 2.25/0.25×10⁶ IFU) were performed by a trans-corneal method at 2 weeks of age to express hRBP3 and luciferase reporter gene. We confirmed luciferase expression in rat eyes at one week, three months and six months after injection using in vivo imaging system (IVIS Lumina system; Caliper Life Sciences, Hopkinton, Mass.) with intraperitoneal injection of D-Luciferin Firefly (50 mg/g BW, Caliper Life Sciences), which correlates well with the expression of RBP3, as detailed below. Diabetes was induced by intraperitoneal injection of streptozotocin (STZ; Sigma-Aldrich, Milwaukee, Wis.; 55 mg/kgBW) after a 12 hrs overnight fast at 6 week of age and ascertained by blood glucose at fasting>250 mg/dl and fed<450mg/dl as measured by a glucometer and followed at 3-4 week intervals. Anesthesia used for these experiments was an intramuscular injection of ketamine (50 mg/kg; Bioniche Pharma, Lake Forest, Ill.) and xylazine (10 mg/kg; Sigma-Aldrich). At the conclusion of the studies, animals were killed by inhalation of carbon dioxide. The experimental protocols were approved by the Joslin Diabetes Center Institutional Animal Care and Use Committee. The experiments were carried out in strict accordance with the recommendations in the Guide for the Care and Use of Laboratory Animals of the National Institutes of Health. The following studies were performed.

### Bioluminescence Imaging

Bioluminescence imaging was performed using an IVIS SpectrumCT Pre-clinical In Vivo Imaging System one week after the subretinal injection to confirm the infection of lentiviral vectors for firefly luciferase into the eyes. Rats were anesthetized by using isoflurane inhalation mixed in pure oxygen followed by an intraperitoneal injection of D-luciferin (50 mg/kg BW). Bioluminescence images in both eyes were acquired 5 min after luciferin inj ection.

### Optical Coherence Tomography

In vivo scanning of the retina was performed at 2 months after STZ injection by using the Bioptigen 840S SD-OCT imaging system (Charlotte, N.C.). Rats were anesthetized by intramuscular injection of ketamine (75-80mg/kg)/xylazine (5-10 mg/kg) prior to the procedure. 2 drops of a dilating agent (1% Tropicamide) were applied to the eye. The rat was placed into a XYZ axis staging cassette that allows for alignment. The image was centered upon the optic nerve. A 2.5×2.5 mm rectangular volume scan was selected consisting of 100 B Scans×1000 A Scans. Each scan of the retina was digitized within 20 seconds. The staging cassette was rotated and the procedure was repeated for the contralateral eye. Rats ware returned to the cages for recovery.

### Elecroretinogram

The elecroretinogram (ERG) recording system (PowerLab, ADlnstruments Santa Clara, Calif.) consists of a light emitting diode light stimulator, amplifiers, a computer, and a display. The light stimulator provides consistent full-field stimulation. The stimulus duration, stimulus intensity, and background intensity were controlled by the LED-driver (WLS-20, Mayo Co., Nagoya, Japan). Maximal stimulus was 1.4×10⁴ cd/m² at the cornea. A white diffuser was placed between the LEDs and cornea to produce a homogenous stimulus and background illumination to the retina. The LED stimulator and contact lens were packaged as a unit and purchased from the vendor supplying the equipment. At 2 months after STZ inj ection, rats were dark-adapted within the ERG room overnight. Under dim red light, the rats were anesthetized with an intramuscular injection of 50 mg/kg ketamine hydrochloride and 10 mg/kg xylazine hydrochloride. The pupils were dilated with 1% tropicamide and kept in a warming box for 10 minutes. Before measurement, rats were placed on an electrically isolated heating pad to maintain the body temperature at 37° C. A drop of Gonak was placed on the eye to maintain contact with the contact lens gold wire electrode. The ERG contact lens was then placed over the cornea. A 29 gauge needle electrode (PowerLab ADlnstruments MLA1202 ERG Needle Electrode), was placed subcutaneously into the base of the tail as a ground. A negative electrode, also a 29 gauge needle electrode, was placed subcutaneously in the forehead of the mouse. For the recording, a white light stimulus intensity of 1.4×10⁴ cd/m² with duration of 5 msec was used. The signals were amplified with a bandpass filter between 1 and 1000Hz (PowerLab ML750) to reduce background noise. At least three signals were recorded with an interval of 20 seconds between stimulations. An average of all signals was used in the data analysis. Analysis of the data was performed using the ADInstruments Scope V3.6.4 software.

### Retinal Vascular Permeability Measurements by Evans Blue-Dye Permeation

The Evans blue-dye permeation technique was performed at 2 months after STZ injection to quantify retinal vascular permeability. In brief, under anesthesia, each rat was infused with Evans blue dye (45 mg/kg BW) through an indwelling jugular catheter. The dye was allowed to circulate for 2 h prior to the time the rats were killed. After tissue fixation, the eyes were enucleated. Retinas were extracted with dimethyl formamide, and the resultant supernatant was used to determine Evans blue-dye content. Results were expressed as the rate of plasma extravasation per unit weight of retinal tissue.

### Western Blot Analyses in Rat Retina

Rats were euthanized as described above, and their eyes were excised and subsequently separated into retina and vitreous which were stored in a deep freezer for the future experiments. Retina was lysed with RIPA buffer including protease inhibitor. Tissue lysates were denatured with x6 sample loading buffer for immunoblotting with the following antibodies: rabbit polyclonal anti-RBP3 (1:2000; ab101456, Abcam), anti-FLAG M2 (1:1000; #2368, Cell Signaling Technology), anti-Myc-Tag (1:1000, #2272, Cell Signaling Technology) and HRP-conjugated anti-β-Actin (Santa Cruz).

### Quantification of Pericyte Loss

We assessed retinal vascular pathology in diabetic rats with subretinal injection of lentiviral vector as reported previously (Geraldes et al., Nature medicine 2009;15:1298-306; Mohr et al., Diabetes 2002;51:1172-9). Whole rat eyes were dissected out at 6 months after STZ injection and fixed in 10% neutral buffered formalin for a week at room temperature. We isolated the retinal vasculature by a trypsin digest method and air-dried the vasculature onto glass slides. We then stained sections of the vasculature with hematoxylin and periodic acid-Schiff, dehydrated them and added coverslips. We quantified acellular capillaries in at least 1,000 capillary cells in four to seven field areas (400×magnification) in the mid-retina in a blinded manner. We identified acellular capillaries as capillary-sized vessel tubes having no nuclei anywhere along their length including or not vessel ‘clusters’, and we reported them per square millimeter of retinal area. To count acellular capillaries, we examined at least 1,000 capillary cells (endothelial cells and pericytes) in five field areas in the mid-retina (400×magnification) in a blinded manner.

### Statistical Analysis

In the human study, we used the Mann-Whitney U test for the assessment of between-group differences in peptide hits in proteomic analysis and quantified band intensities of immunoblotting for vitreous RBP3. Data were reported as medians and interquartile ranges. For in vitro and in vivo studies, we used the unpaired two-tailed t-test for the comparison of groups. In physiological analysis of Lewis rats, eyes with dense cataracts or whole corneal defect were excluded from statistical analysis because of unreliability of the results due to the disturbance of optical clearance. Data were reported as means and standard deviations. A p-value of less than 0.05 was considered to indicate statistical significance.

### Example 1. Factors Protective against Diabetic Retinopathy In Human Eye

To identify proteins increased in the retinal vascular unit and intraocular space as potential candidates for protective factors against DR, human retina and vitreous samples were analyzed by a LC/MS-based protein quantification method. Clinical characteristics of 83 participants in the current study used in proteomics and western blot analysis according to DR grade were described in Table 1. Overall major traits in subjects with diabetes were, age 66 (56-78), female gender 57.7%, type 1 diabetes 62.0%, HbA1c 7.4 (6.9-8.4) %, eGFR 54.2 (40.5-67.9) ml/min/1.73m² and CVD 44.9%, indicated as medians (interquartile range) or percentages. Proteomic analysis using 17 eyes from 10 Medalists identified an exclusive peptide highly increased in both retina and vitreous of 6 no-mild NPDR compared to those of 11 QPDR; RBP3, interphotoreceptor retinoid-binding protein, was the only peptide that filled the criteria (1.5-fold increase in no-mild NPDR vs. QPDR with p-value less than 0.05 in both retina and vitreous) out of 318 retina and 327 vitreous detected peptides (Table 2). Peptide numbers of RBP3 were increased by 1.6-fold in retina (p=0.04) and by 2.1-fold in vitreous (p=0.005) of Medalists with no-mild NPDR compared to those with QPDR. RBP3 expression levels in vitreous of mixed population including subjects with type 1 diabetes (Medalists and non-Medalists) and type 2 diabetes according to DR grades and NDM controls were determined by immunoblotting (FIG. 1A) to support proteomics result. Vitreous RBP3 expression in QPDR (−45% to NPDR, p=0.05) and APDR (−36% to NPDR, p=0.03) were lower than NPDR, while there was no significant change between NPDR and NDM. Quality of immunoblotting was shown in FIG. 1B.

TABLE 1
Clinical Characteristics by Disease Status Used in Proteomics and Western Blot Analysis.

| | Non-DM | Non-PDR | Quiescent PDR | Active PDR |
|---|---|---|---|---|
| Total number | 12 | 21 | 18 | 32 |
| Number of Medalists | N/A | 14 | 15 | N/A |
| | Percentage or Median [lower quartile, upper quartile] | | | |
| Age (years) | 79 [73, 83] | 80 [71, 82.5] | 71.5 [63.8, 79.3] | 60 [43.5, 66] |
| Gender (Female, %) | 41.7% | 52.4% | 33.3% | 40.6% |
| Type of diabetes | N/A | 66.7% | 94.4% | 40.6% |
| (Type 1, %) | | | | |
| Duration of disease | N/A | 60 [27, 72]^{(2†)} | 58 [51, 72.5] | 24.5 [18.5, 30] |
| (years) | | | | |
| Age at diagnosis | N/A | 15.5 [5.3, 22.8]^{(5†)} | 8 [5.5, 11.5]^{(1†)} | 32 [13, 45.5] |
| (years) | | | | |
| HbA1c (%) | N/A | 7.1 [6.8, 8.2]^{(2†)} | 7.5 [7.0, 8.3] | 7.8 [7.1, 9.1]^{(8†)} |
| BMI (kg/m²)* | N/A | 23.2 [21.4, | 24.7 [21.5, 28.9] | N/A |
| | | 26.5] | | |
| Total Cholesterol | N/A | 155 [135, 178]^{(5†)} | 158 [141, 168]^{(1†)} | 186 [129, 210]^{(11†)} |
| (mg/dL) | | | | |
| Triglycerides (mg/dL) | N/A | 71 [55, 156]^{(4†)} | 69 [47, 115] | 90 [58, 202]^{(12†)} |
| HDL (mg/dL) | N/A | 60 [46, 72]^{(5†)} | 54 [45, 74]^{(1†)} | 44 [36, 62]^{(11†)} |
| LDL (mg/dL) | N/A | 71 [56, 97]^{(5†)} | 73 [65.5, 91]^{(1†)} | 102 [59, 127]^{(11†)} |
| CRP (mg/L)* | N/A | 0.8 [0.52, 3.05]^{(1†)} | 2.1 [0.91, 6.6] | N/A |
| eGFR (ml/min/ | N/A | 57.5 [40.8, | 49.8 [39.3, 71.8] | N/A |
| 1.73 m²)* | | 67.0] | | |
| Hypertension (%) | 33.3%^{(3†)} | 57.9%^{(2†)} | 56.3%^{(2†)} | N/A |
| Neuropathy (%) | N/A | 42.1%^{(2†)} | 66.7% | 31.3% |
| CVD (%) | N/A | 52.6%^{(2†)} | 83.3% | 18.8% |

DM = diabetes mellitus.
PDR = proliferative diabetic retinopathy.
BMI = body mass index.
HDL = high-density lipoprotein cholesterol.
LDL = low-density lipoprotein cholesterol.
CRP = C-reactive protein.
eGFR = estimated glomerular filtration rate.
CVD = cardiovascular disease.
*Available only in Medalists.
^{†}Number of lacking data.

TABLE 2
A peptide selected by proteomic analysis in retina and vitreous of Medalists

| Protein Name | Retina n=17 eyes, 10 people | | | | Vitreous n=17 eyes, 10 people | | | |
|---|---|---|---|---|---|---|---|---|
| RBP3 Interphotoreceptor | No-Mild NPDR | QPDR | | | No-Mild NPDR | QPDR | | |
| retinoid-binding protein | n = 6 | n = 11 | | | n = 6 | n = 11 | | |
| MW: 135366 Da Accession | eyes, 4 | eyes, 6 | Fold - | P- | eyes, 4 | eyes, 6 | Fold - | P- |
| number: IPI00022337.1 | people | people | change | value | people | people | change | value |
| Peptide hit numbers (Median) | 414.5 | 268 | 1.55 | 0.04 | 560 | 269 | 2.08 | <0.01 |

The proteomic analyses in retina and vitreous were performed as described in the previous report (Gao B B, Chen X, Timothy N, Aiello L P, Feener E P. Characterization of the vitreous proteome in diabetes without diabetic retinopathy and diabetes with proliferative diabetic retinopathy. J Proteome Res 2008 Jun; 7(6): 2516-25). Total numbers of detected peptides were 318 (retina) and 327 (vitreous) in median of all subjects. The selecting criteria was a minimum 1.5-fold increase of peptide numbers with P-value < 0.05 in both retina and vitreous. P-values were calculated by Kruskal-Wallis test.

### Example 2. Characterization of Human RBP3 Function in Retinal Vascular Cells

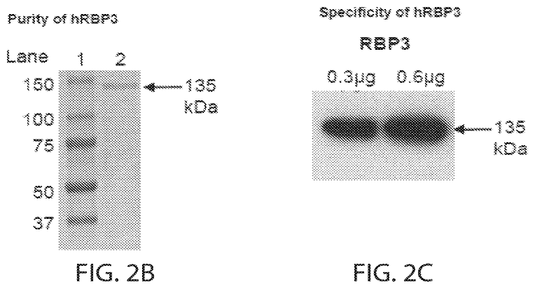

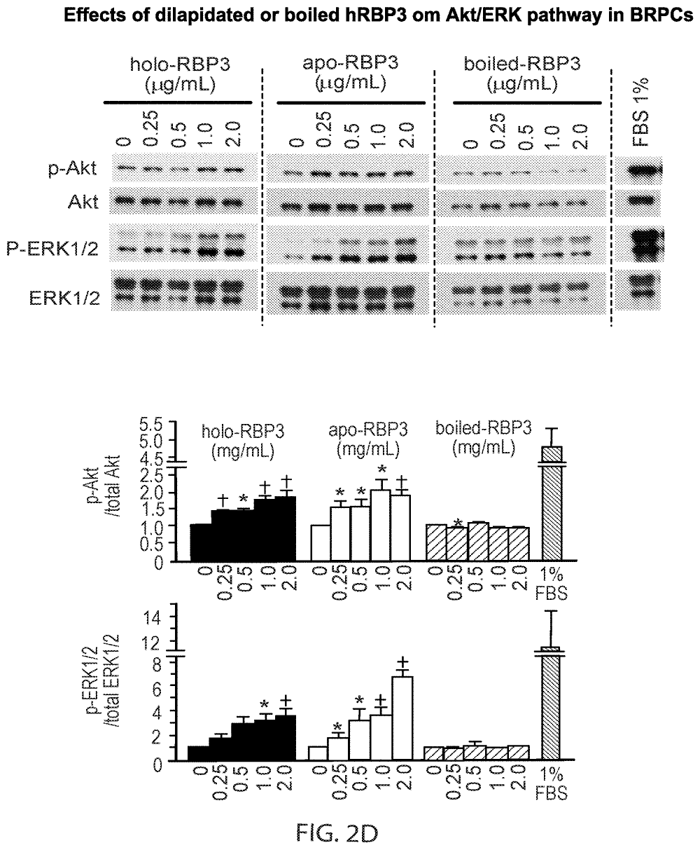

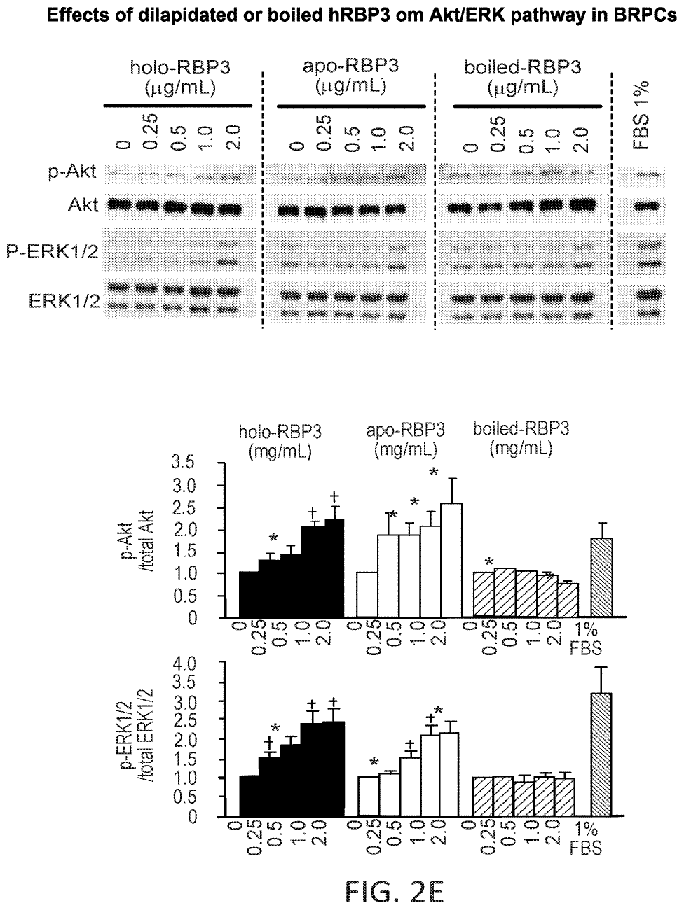

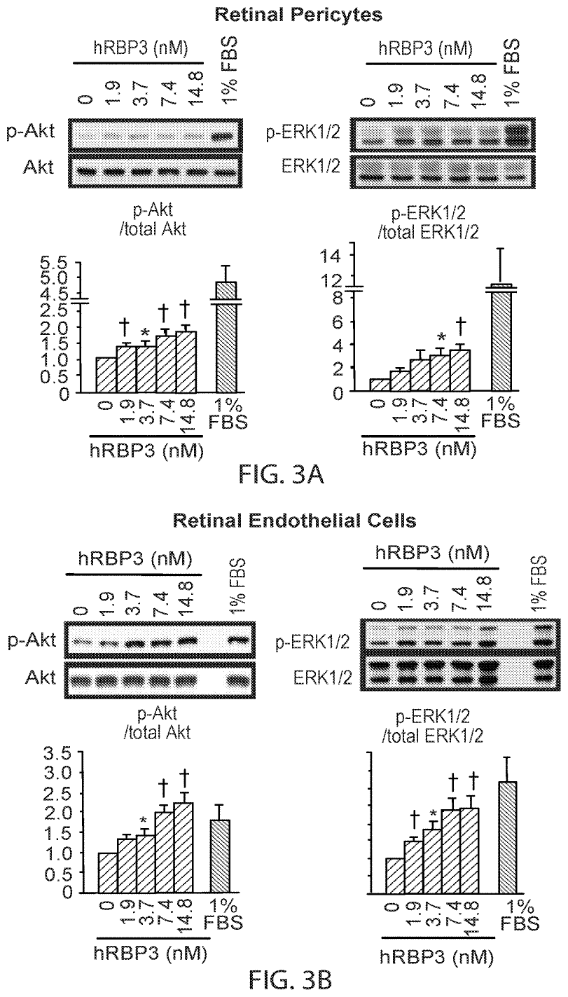

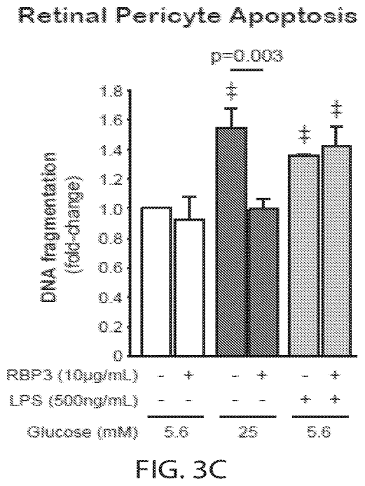

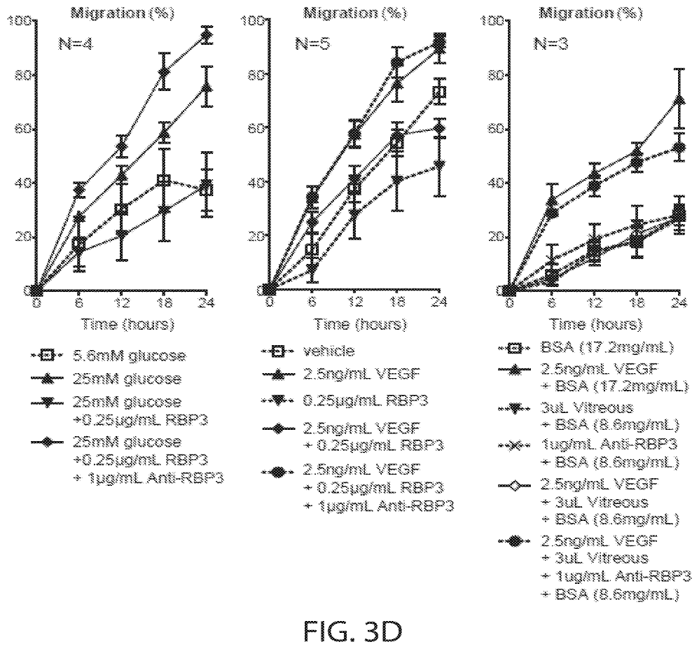

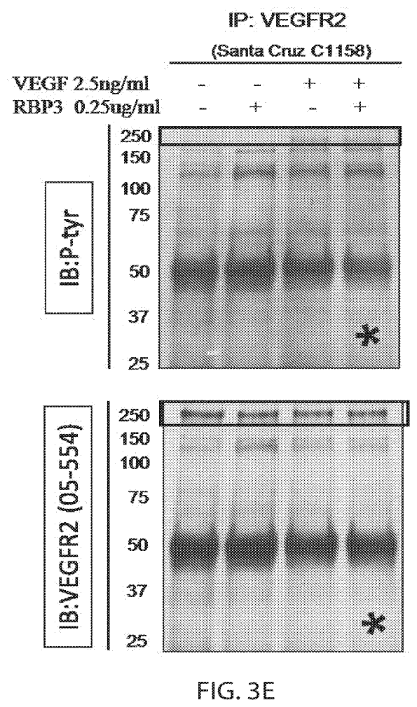

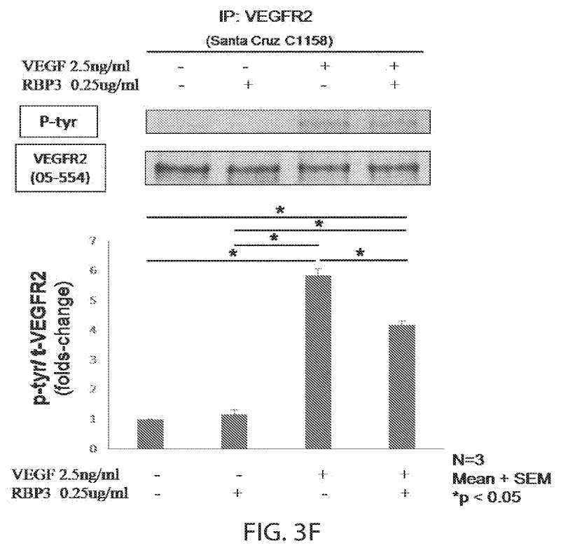

To test the effects of human RBP3 (hRBP3) on retinal vascular cells, hRBP3 was extracted from the serum free culture media of 293A cells after a large-scale transient transfection with CMV-promoted expression vector (FIGS. 2A, B and C). Retinoic acid was detected in holo-RBP3 (2.0 ng/mL) but not in delipidated apo-RBP3 (Table 3). Phosphorylation of Akt (Ser⁴⁷³) and ERK1/2 (Thr²⁰²/Tyr²⁰⁴) in bovine retinal pericytes (BRPCs) and endothelial cells (BRECs) were significantly stimulated by short exposure (for 10 min) to hRBP3 in a dose-dependent manner (FIGS. 3A-3B). Delipidated apo-RBP3 stimulates phosphorylation of Akt and ERK in the same manner to holo-RBP3 in both BRPCs (FIG. 3A) and BRECs (FIG. 3B), while inactivated RBP3 by boiling did not. Retinal pericyte apoptosis induced by high glucose condition (25 mM) was completely inhibited by 10 μg/mL (=14.8 nM) of hRBP3, while LPS-induced apoptosis was not (FIG. 3C). Retinal endothelial cell migrations induced by high glucose (25 mM) and VEGF (2.5 ng/mL) were inhibited by hRBP3 (0.25 μg/mL=1.9 nM) to the basal levels with significance. Medalist vitreous with high RBP3 expression graded as NPDR, adjusted to the same RBP3 concentration to hRBP3 experiments (0.25 μg/mL) according to the result from western blot analysis (FIGS. 1A and 1B), also completely inhibited VEGF-induced endothelial cell migration (FIG. 3D). Neutralizing antibody for RBP3 completely reversed these inhibitory effects of hRBP3 on migration of BRECs to the level of each stimulant, although there was no change when added to basal condition. When co-administered, RBP3 had an inhibitory effect on VEGF-induced pFlk-migration in BREC (3E-F).

TABLE 3
Efficacy of delipidation of hRBP3

| | Halo-RBP3 | |
|---|---|---|
| | (Retinol- | Apo-RBP3 |
| | bound) | (Delipidated) |
| Content of retinoic acid | 2.03 ng/mL | not detected |
| Protein concentration | 0.27 mg/mL | 0.28 mg/mL |

Table 3 shows the profiles of hRBP3 before and after delipidation, which guaranteed the removal of retinoic acid without changing protein concentration measured by BCA protein assay.

### Example 3. Human RBP3 Effects on Neuroretinal Dysfunction in Diabetes

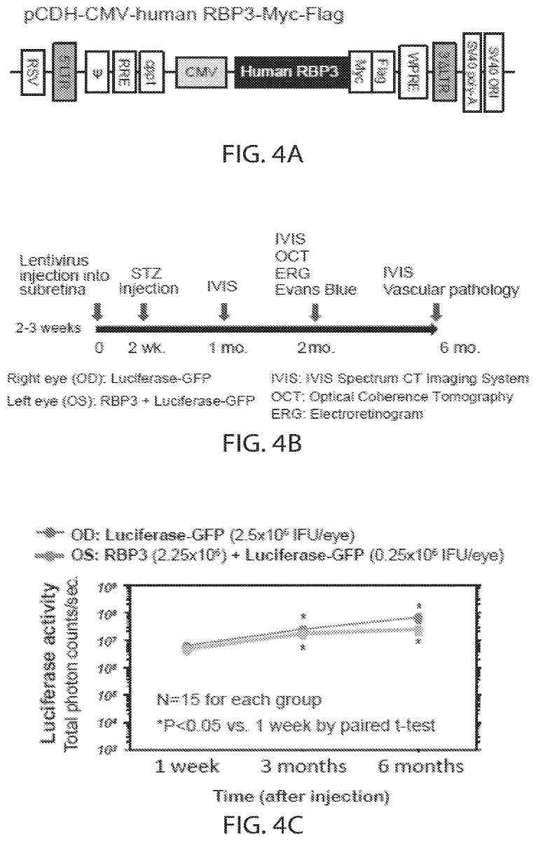

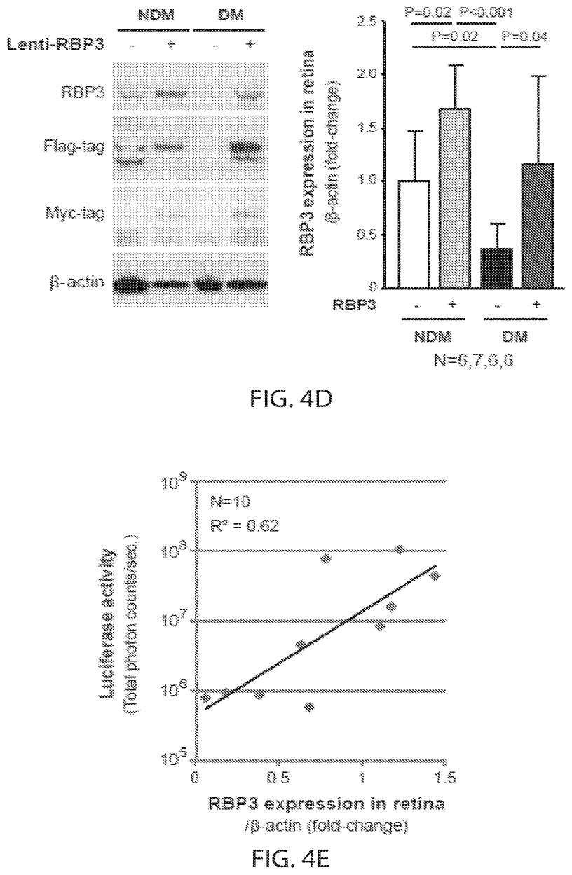

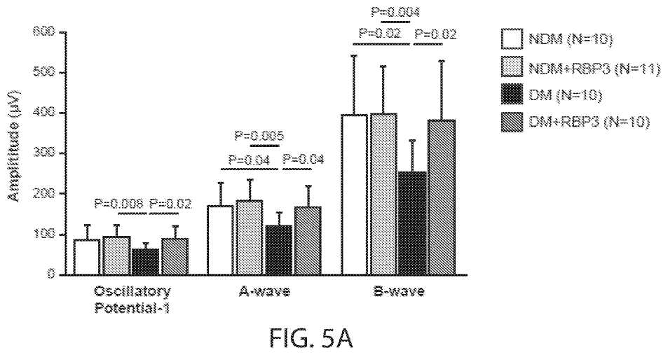

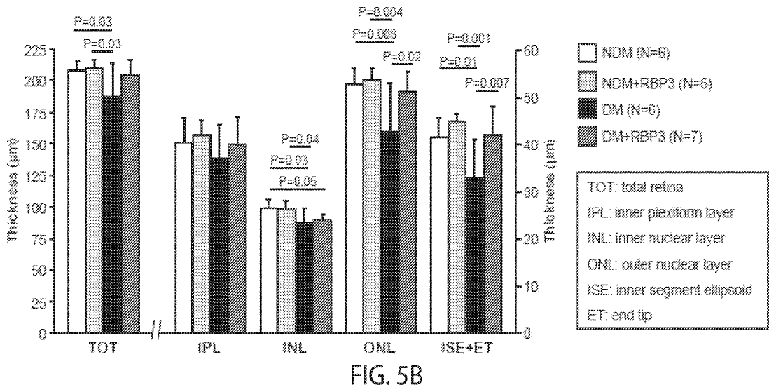

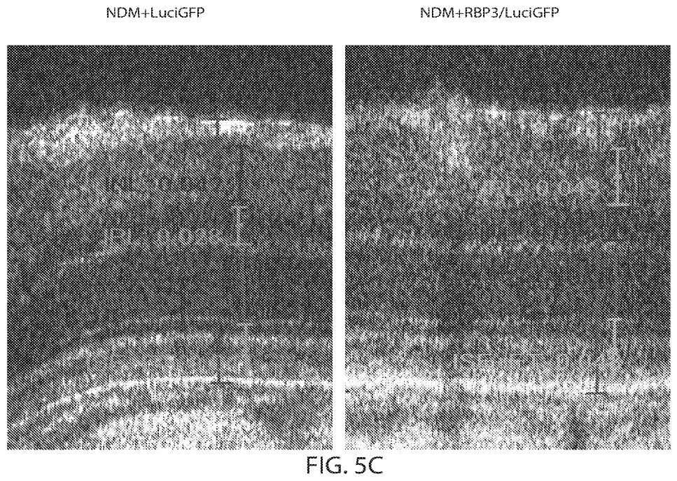

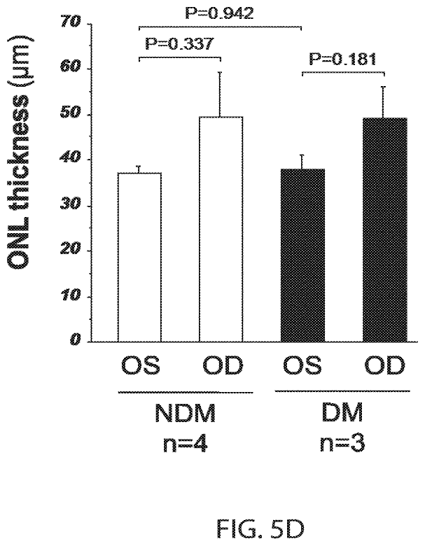

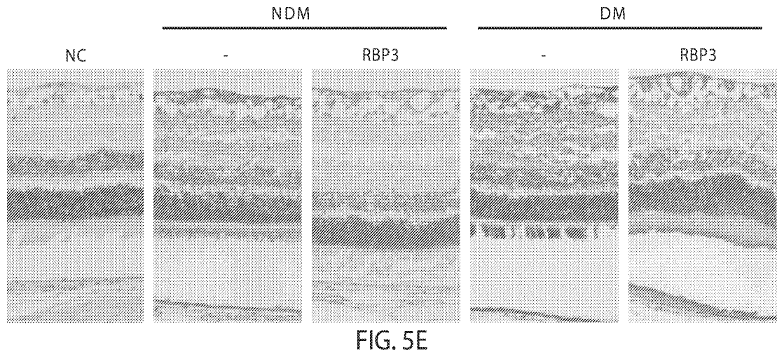

Lentiviral vector expressing human RBP3 transgene (FIG. 4A) was injected into subretina of Lewis rats, a strain prone to neurodegeneration and loss of retinal capillaries,²⁰ to investigate the role of RBP3 in diabetic retinopathy. Functional and structural changes of neuroretina were assessed by ERG and OCT at 1 month after the induction of diabetes by STZ, respectively (FIG. 4B). RBP3 expression was determined in combination of co-expressed luciferase activity detected by minimally invasive in vivo imaging system for time-course and postmortem western blot analysis (FIG. 4C). The model showed consistent RBP3 expression throughout the experimental term in the eyeground. RBP3 expression in retina was correlated to IVIS signal and significantly decreased in diabetic rats, recovered by exogenous RBP3 with Myc/FLAG-tag to non-diabetic controls (FIGS. 4D and 4E). As shown in FIG. 5A, the significant decrease of amplitudes of oscillatory potential 1, A-wave and B-wave in response to light stimuli by ERG designated impaired function of neural retina in diabetic rats. RBP3 overexpression in retina rescued the neuronal dysfunction to non-diabetic control level. FIGS. 5B-E shows the thinner thicknesses of total retina and retinal sub-layers measured by OCT in normal rats compared to controls, which were increased by RBP3 overexpression especially in photoreceptor layers (ONL and ISE+ET).

### Example 4. Protective Role of Human RBP3 in Retina

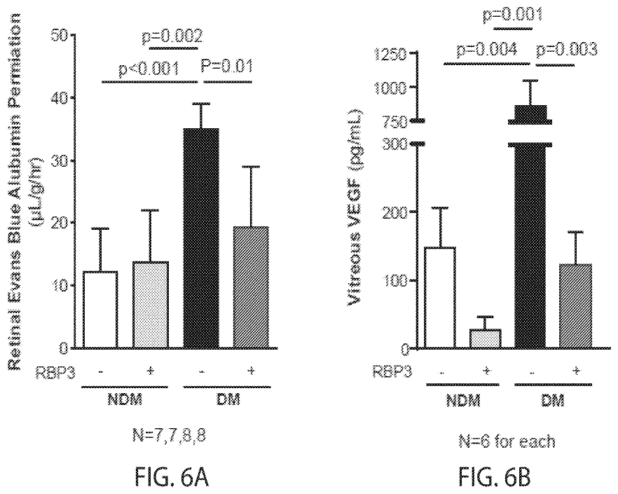

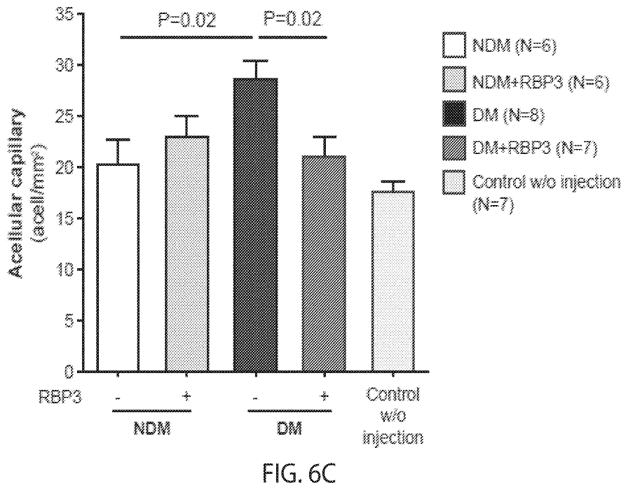

Retinal vascular dysfunction in normal and diabetic rats were assessed by retinal vascular permeability along with VEGF expression at 2 months and acellular capillaries in retinal vascular pathology at 6 months after induction of diabetes using the same experimental animals with RBP3 overexpression in subretina (FIG. 4B). Vascular permeability was significantly increased by 2.9-fold in diabetic rats compared to non-diabetic rats, which was reduced by RBP3 overexpression (−69% of the increase), but not significantly changed in normal animals (FIG. 6A). At the same time point, VEGF levels in vitreous increased by 5.8-fold in DM was completely reduced by RBP3 (−103% of the increase) and a similar decrease in VEGF levels was seen in normal animals overexpressing VEGF (FIG. 6B). The number of acellular capillaries, a morphometric marker of early diabetic retinopathy, was significantly increased by 1.4-fold in 6-Mo DM compared to age-matched NDM, which was reversed by RBP3 to the NDM level (−92% of the increase), while no significant change was seen in NDM animals (FIG. 6C).

### References

1. Bourne et al. Causes of vision loss worldwide, 1990-2010: a systematic analysis. The Lancet Global health 2013;1:e339-49.

2. Aiello et al. Vascular endothelial growth factor in ocular fluid of patients with diabetic retinopathy and other retinal disorders. The New England journal of medicine 1994;331:1480-7.

3. Aiello et al. Suppression of retinal neovascularization in vivo by inhibition of vascular endothelial growth factor (VEGF) using soluble VEGF-receptor chimeric proteins. Proceedings of the National Academy of Sciences of the United States of America 1995;92:10457-61.

4. Diabetic Retinopathy Clinical Research N, Elman M J, Aiello L P, et al. Randomized trial evaluating ranibizumab plus prompt or deferred laser or triamcinolone plus prompt laser for diabetic macular edema. Ophthalmology 2010;117:1064-77 e35.

5. Geraldes et al. Activation of PKC-delta and SHP-1 by hyperglycemia causes vascular cell apoptosis and diabetic retinopathy. Nature medicine 2009;15:1298-306.

6. Kondo and Kahn, Altered insulin signaling in retinal tissue in diabetic states. The Journal of biological chemistry 2004;279:37997-8006.

7. Reiter et al. Diabetes reduces basal retinal insulin receptor signaling: reversal with systemic and local insulin. Diabetes 2006;55:1148-56.

8. Mizutani et al. Accelerated death of retinal microvascular cells in human and experimental diabetic retinopathy. The Journal of clinical investigation 1996;97:2883-90.

9. Cogan et al. Retinal vascular patterns. IV. Diabetic retinopathy. Archives of ophthalmology 1961;66:366-78.

10. Barber et al. Neural apoptosis in the retina during experimental and human diabetes. Early onset and effect of insulin. The Journal of clinical investigation 1998;102:783-91.

11. van Dijk et al. Selective loss of inner retinal layer thickness in type 1 diabetic patients with minimal diabetic retinopathy. Investigative ophthalmology & visual science 2009;50:3404-9.

12. Bearse et al. Local multifocal oscillatory potential abnormalities in diabetes and early diabetic retinopathy. Investigative ophthalmology & visual science 2004;45:3259-65.

13. Bresnick and Palta. Predicting progression to severe proliferative diabetic retinopathy. Archives of ophthalmology 1987;105:810-4.

14. Antonetti et al. Diabetic retinopathy. The New England journal of medicine 2012;366:1227-39.

15. Gao et al. Characterization of the vitreous proteome in diabetes without diabetic retinopathy and diabetes with proliferative diabetic retinopathy. Journal of proteome research 2008;7:2516-25.

16. Cunha-Vaz. Studies on the pathophysiology of diabetic retinopathy. The blood-retinal barrier in diabetes. Diabetes 1983;32 Suppl 2:20-7.

17. Sun et al. Protection from retinopathy and other complications in patients with type 1 diabetes of extreme duration: the joslin 50-year medalist study. Diabetes care 2011;34:968-74.

18. Keenan et al. Residual insulin production and pancreatic ss-cell turnover after 50 years of diabetes: Joslin Medalist Study. Diabetes 2010;59:2846-53.

19. Nayak et al. A monoclonal antibody (3G5)-defined ganglioside antigen is expressed on the cell surface of microvascular pericytes. The Journal of experimental medicine 1988;167:1003-15.

20. Kern et al. Comparison of three strains of diabetic rats with respect to the rate at which retinopathy and tactile allodynia develop. Molecular vision 2010;16:1629-39.

21. Chader. Interphotoreceptor retinoid-binding protein (IRBP): a model protein for molecular biological and clinically relevant studies. Friedenwald lecture. Investigative ophthalmology & visual science 1989;30:7-22.

22. Fong et al. Purification and characterization of a retinol-binding glycoprotein synthesized and secreted by bovine neural retina. The Journal of biological chemistry 1984;259:6534-42.

23. Fong et al. Characterization, localization, and biosynthesis of an interstitial retinol-binding glycoprotein in the human eye. Journal of neurochemistry 1984;42:1667-76.

24. Wiggert et al. Immunochemical distribution of interphotoreceptor retinoid-binding protein in selected species. Investigative ophthalmology & visual science 1986;27:1041-9.

25. Salvador-Silva et al. Retinoid processing proteins in the ocular ciliary epithelium. Molecular vision 2005;11:356-65.

26. Garcia-Ramirez et al. Interphotoreceptor retinoid-binding protein (IRBP) is downregulated at early stages of diabetic retinopathy. Diabetologia 2009;52:2633-41.

27. DesJardin et al. Transcription of photoreceptor genes during fetal retinal development. Evidence for positive and negative regulation. The Journal of biological chemistry 1993;268:6953-60.

28. Ho et al. Mechanism of vitamin A movement between rod outer segments, interphotoreceptor retinoid-binding protein, and liposomes. The Journal of biological chemistry 1989;264:928-35.

29. den Hollander et al. A homozygous missense mutation in the IRBP gene (RBP3) associated with autosomal recessive retinitis pigmentosa. Investigative ophthalmology & visual science 2009;50:1864-72.

30. Ksantini et al. Screening genes of the visual cycle RGR, RBP1 and RBP3 identifies rare sequence variations. Ophthalmic genetics 2010;31:200-4.

31. Bridges et al. Interstitial retinol-binding protein (IRBP) in subretinal fluid. Investigative ophthalmology & visual science 1986;27:1027-30.

32. Newsome et al. Interphotoreceptor retinoid-binding protein levels in subretinal fluid from rhegmatogenous retinal detachment and retinopathy of prematurity. Archives of ophthalmology 1988;106:106-10.

33. Wisard et al. Exaggerated eye growth in IRBP-deficient mice in early development. Investigative ophthalmology & visual science 2011;52:5804-11.

34. Sato et al. Receptor interacting protein kinase-mediated necrosis contributes to cone and rod photoreceptor degeneration in the retina lacking interphotoreceptor retinoid-binding protein. The Journal of neuroscience : the official journal of the Society for Neuroscience 2013;33:17458-68.

35. Ripps et al. The rhodopsin cycle is preserved in IRBP “knockout” mice despite abnormalities in retinal structure and function. Visual neuroscience 2000;17:97-105.

36. Geraldes et al. Selective regulation of heme oxygenase-1 expression and function by insulin through IRS1/phosphoinositide 3-kinase/Akt-2 pathway. The Journal of biological chemistry 2008;283:34327-36.

37. Bazan et al.. Endogenous fatty acids are covalently and noncovalently bound to interphotoreceptor retinoid-binding protein in the monkey retina. The Journal of biological chemistry 1985;260:13677-80.

38. Suzuma et al. Stretch-induced retinal vascular endothelial growth factor expression is mediated by phosphatidylinositol 3-kinase and protein kinase C (PKC)-zeta but not by stretch-induced ERK1/2, Akt, Ras, or classical/novel PKC pathways. The Journal of biological chemistry 2002;277:1047-57.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of increasing retinal thickness in a non-diabetic mammal, the method comprising administering to the mammal one or both of:
(i) a composition comprising RBP3 polypeptide, and/or
(ii) a composition comprising a nucleic acid encoding an RBP3 polypeptide.

2. (canceled)

3. The method of claim 1, wherein the mammal is a human,

4. The method of claim 1, wherein the mammal has a retinal degenerative disorders associated with retinal thinning.

5. The method of claim 4, wherein the disorder is retinitis pigmentosa, lattice degeneration, or Stargardt disease.

6. The method of claim 4, wherein the disorder is retinal thinning associated with multiple sclerosis or Gaucher's disease.

7. The method of claim 1, wherein the nucleic acid encoding an RBP3 polypeptide is in a viral vector.

8. The method of claim 7, wherein the viral vector is an adeno-associated virus or a lentivirus.

9. The method of claim 1, wherein the nucleic acid encoding an RBP3 polypeptide comprises a sequence that is at least 80% identical to nucleotides 1 to 4276, 1 to 3855, 115 to 4276, 115 to 3855, 166 to 3855, 166 to 4276, 1 to 4151, 115 to 4151, or 166 to 4151 of SEQ ID NO:2.

10. The method of claim 1, wherein the RBP3 polypeptide comprises a sequence that is at least 80% identical to amino acids 18-1247 of SEQ ID NO:1.

11. The method or composition of claim 1, wherein the composition is administered by local (ocular) administration.

12. The method of claim 1, wherein the composition is formulated for administration on, in, or into the eye.

13. The method of claim 1, wherein the composition is formulated in eye drops, lotions, creams, or ointment.


SEQUENCE LISTING (2 sequences)

SEQ ID NO: 1 - protein, 2494 aa, Homo sapiens
MMREWVLLMSVLLCGLAGPTHLFQPSLVLDMAKVLLDNYCFPENLLGMQEAIQQAIKSHEILSISDPQTLASVLTAGVQSSLNDPRLVISYEPSTPEPPPQVPALTSLSEEELLAWLQRGLRHEVLEGNVGYLRVDSVPGQEVLSMMGEFLVAHVWGNLMGTSALVLDLRHCTGGQVSGIPYIISYLHPGNTILHVDTIYNRPSNTTTEIWTLPQVLGERYGADKDVVVLTSSQTRGVAEDIAHILKQMRRAIVVGERTGGGALDLRKLRIGESDFFFTVPVSRSLGPLGGGSQTWEGSGVLPCVGTPAEQALEKALAILTLRSALPGVVHCLQEVLKDYYTLVDRVPTLLQHLASMDFSTVVSEEDLVTKLNAGLQAASEDPRLLVRAIGPTETPSWPAPDAAAEDSPGVAPELPEDEAIRQALVDSVFQVSVLPGNVGYLRFDSFADASVLGVLAPYVLRQVWEPLQDTEHLIMDLRHNPGGPSSAVPLLLSYFQGPEAGPVHLFTTYDRRTNITQEHFSHMELPGPRYSTQRGVYLLTSHRTATAAEEFAFLMQSLGWATLVGEITAGNLLHTRTVPLLDTPEGSLALTVPVLTFIDNHGEAWLGGGVVPDAIVLAEEALDKAQEVLEFHQSLGALVEGTGHLLEAHYARPEVVGQTSALLRAKLAQGAYRTAVDLESLASQLTADLQEVSGDHRLLVFHSPGELVVEEAPPPPPAVPSPEELTYLIEALFKTEVLPGQLGYLRFDAMAELETVKAVGPQLVRLVWQQLVDTAALVIDLRYNPGSYSTAIPLLCSYFFEAEPRQHLYSVFDRATSKVTEVWTLPQVAGQRYGSHKDLYILMSHTSGSAAEAFAHTMQDLQRATVIGEPTAGGALSVGIYQVGSSPLYASMPTQMAMSATTGKAWDLAGVEPDITVPMSEALSIAQDIVALRAKVPTVLQTAGKLVADNYASAELGAKMATKLSGLQSRYSRVTSEVALAEILGADLQMLSGDPHLKAAHIPENAKDRIPGIVPMQIPSPEVFEELIKFSFHTNVLEDNIGYLRFDMFGDGELLTQVSRLLVEHIWKKIMHTDAMIIDMRFNIGGPTSSIPILCSYFFDEGPPVLLDKIYSRPDDSVSELWTHAQVVGERYGSKKSMVILTSSVTAGTAEEFTYIMKRLGRALVIGEVTSGGCQPPQTYHVDDTNLYLTIPTARSVGASDGSSWEGVGVTPHVVVPAEEALARAKEMLQHNQLRVKRSPGLQDHLMMREWVLLMSVLLCGLAGPTHLFQPSLVLDMAKVLLDNYCFPENLLGMQEAIQQAIKSHEILSISDPQTLASVLTAGVQSSLNDPRLVISYEPSTPEPPPQVPALTSLSEEELLAWLQRGLRHEVLEGNVGYLRVDSVPGQEVLSMMGEFLVAHVWGNLMGTSALVLDLRHCTGGQVSGIPYIISYLHPGNTILHVDTIYNRPSNTTTEIWTLPQVLGERYGADKDVVVLTSSQTRGVAEDIAHILKQMRRAIVVGERTGGGALDLRKLRIGESDFFFTVPVSRSLGPLGGGSQTWEGSGVLPCVGTPAEQALEKALAILTLRSALPGVVHCLQEVLKDYYTLVDRVPTLLQHLASMDFSTVVSEEDLVTKLNAGLQAASEDPRLLVRAIGPTETPSWPAPDAAAEDSPGVAPELPEDEAIRQALVDSVFQVSVLPGNVGYLRFDSFADASVLGVLAPYVLRQVWEPLQDTEHLIMDLRHNPGGPSSAVPLLLSYFQGPEAGPVHLFTTYDRRTNITQEHFSHMELPGPRYSTQRGVYLLTSHRTATAAEEFAFLMQSLGWATLVGEITAGNLLHTRTVPLLDTPEGSLALTVPVLTFIDNHGEAWLGGGVVPDAIVLAEEALDKAQEVLEFHQSLGALVEGTGHLLEAHYARPEVVGQTSALLRAKLAQGAYRTAVDLESLASQLTADLQEVSGDHRLLVFHSPGELVVEEAPPPPPAVPSPEELTYLIEALFKTEVLPGQLGYLRFDAMAELETVKAVGPQLVRLVWQQLVDTAALVIDLRYNPGSYSTAIPLLCSYFFEAEPRQHLYSVFDRATSKVTEVWTLPQVAGQRYGSHKDLYILMSHTSGSAAEAFAHTMQDLQRATVIGEPTAGGALSVGIYQVGSSPLYASMPTQMAMSATTGKAWDLAGVEPDITVPMSEALSIAQDIVALRAKVPTVLQTAGKLVADNYASAELGAKMATKLSGLQSRYSRVTSEVALAEILGADLQMLSGDPHLKAAHIPENAKDRIPGIVPMQIPSPEVFEELIKFSFHTNVLEDNIGYLRFDMFGDGELLTQVSRLLVEHIWKKIMHTDAMIIDMRFNIGGPTSSIPILCSYFFDEGPPVLLDKIYSRPDDSVSELWTHAQVVGERYGSKKSMVILTSSVTAGTAEEFTYIMKRLGRALVIGEVTSGGCQPPQTYHVDDTNLYLTIPTARSVGASDGSSWEGVGVTPHVVVPAEEALARAKEMLQHNQLRVKRSPGLQDHL

SEQ ID NO: 2 - protein, 4289 aa, Homo sapiens
TGTCCACCAGCTGAGAAGGACAAGGGCGGAAGGCAGCTGCACAGAGCAGGGCCACGGCCTTGCACACAGTCCAGGGAGCTTTTGTGCAGGAGCCAGGCCTCCCCCTGGGTCCCCATGATGAGAGAATGGGTTCTGCTCATGTCCGTGCTGCTCTGTGGCCTGGCTGGCCCCACACACCTGTTCCAGCCAAGCCTGGTGCTGGACATGGCCAAGGTCCTCTTGGATAACTACTGCTTCCCGGAGAACCTGCTGGGCATGCAGGAAGCCATCCAGCAGGCCATCAAGAGCCATGAGATTCTGAGCATCTCAGACCCGCAGACGCTGGCCAGTGTGCTGACAGCCGGGGTGCAGAGCTCCCTGAACGATCCTCGCCTGGTCATCTCCTATGAGCCCAGCACCCCCGAGCCTCCCCCACAAGTCCCAGCACTCACCAGCCTCTCAGAAGAGGAACTGCTTGCCTGGCTGCAAAGGGGCCTCCGCCATGAGGTTCTGGAGGGTAATGTGGGCTACCTGCGGGTGGACAGCGTCCCGGGCCAGGAGGTGCTGAGCATGATGGGGGAGTTCCTGGTGGCCCACGTGTGGGGGAATCTCATGGGCACCTCCGCCTTAGTGCTGGATCTCCGGCACTGCACAGGAGGCCAGGTCTCTGGCATTCCCTACATCATCTCCTACCTGCACCCAGGGAACACCATCCTGCACGTGGACACTATCTACAACCGCCCCTCCAACACCACCACGGAGATCTGGACCTTGCCCCAGGTCCTGGGAGAAAGGTACGGTGCCGACAAGGATGTGGTGGTCCTCACCAGCAGCCAGACCAGGGGCGTGGCCGAGGACATCGCGCACATCCTTAAGCAGATGCGCAGGGCCATCGTGGTGGGCGAGCGGACTGGGGGAGGGGCCCTGGACCTCCGGAAGCTGAGGATAGGCGAGTCTGACTTCTTCTTCACGGTGCCCGTGTCCAGGTCCCTGGGGCCCCTTGGTGGAGGCAGCCAGACGTGGGAGGGCAGCGGGGTGCTGCCCTGTGTGGGGACTCCGGCCGAGCAGGCCCTGGAGAAAGCCCTGGCCATCCTCACTCTGCGCAGCGCCCTTCCAGGGGTAGTCCACTGCCTCCAGGAGGTCCTGAAGGACTACTACACGCTGGTGGACCGTGTGCCCACCCTGCTGCAGCACTTGGCCAGCATGGACTTCTCCACGGTGGTCTCCGAGGAAGATCTGGTCACCAAGCTCAATGCCGGCCTGCAGGCTGCGTCTGAGGATCCCAGGCTCCTGGTGCGAGCCATCGGGCCCACAGAAACTCCTTCTTGGCCCGCGCCCGACGCTGCAGCCGAAGACTCACCAGGGGTGGCCCCAGAGTTGCCTGAGGACGAGGCTATCCGGCAAGCACTGGTGGACTCTGTGTTCCAGGTGTCGGTGCTGCCAGGCAATGTGGGCTACCTGCGCTTCGATAGTTTTGCTGACGCCTCCGTCCTGGGTGTGTTGGCCCCATATGTCCTGCGCCAGGTGTGGGAGCCGCTACAGGACACGGAGCACCTCATCATGGACCTGCGCCACAACCCTGGAGGGCCATCCTCTGCTGTGCCCCTGCTCCTGTCCTACTTCCAGGGCCCTGAGGCCGGCCCCGTGCACCTCTTCACCACCTATGATCGCCGCACCAACATCACGCAGGAGCACTTCAGCCACATGGAGCTCCCGGGCCCACGCTACAGCACCCAACGTGGGGTGTATCTGCTCACCAGCCACCGCACCGCCACGGCCGCGGAGGAGTTCGCCTTCCTTATGCAGTCGCTGGGCTGGGCCACACTGGTAGGTGAGATCACCGCGGGCAACCTGCTGCACACCCGCACGGTGCCGCTGCTGGACACACCCGAAGGCAGCCTCGCGCTCACCGTGCCGGTCCTCACCTTCATCGACAATCACGGCGAGGCCTGGCTGGGTGGTGGAGTGGTGCCCGATGCCATCGTGCTGGCCGAGGAGGCCCTGGACAAAGCCCAGGAAGTGCTGGAGTTCCACCAAAGCCTGGGGGCCTTGGTGGAGGGCACAGGGCACCTGCTGGAGGCCCACTATGCTCGGCCAGAGGTCGTGGGGCAGACCAGTGCCCTCCTGCGGGCCAAGCTGGCCCAGGGCGCCTACCGCACAGCTGTGGACTTGGAGTCTCTGGCCTCTCAGCTCACAGCAGACCTCCAGGAGGTGTCTGGGGACCACCGCTTGCTAGTGTTCCACAGCCCTGGCGAGCTGGTGGTAGAGGAAGCACCCCCACCACCCCCTGCTGTCCCCTCTCCAGAGGAGCTCACCTACCTTATTGAGGCCCTGTTCAAGACAGAGGTGCTGCCCGGCCAGCTGGGCTACCTGCGTTTTGACGCCATGGCTGAACTGGAGACAGTGAAGGCCGTGGGGCCACAGCTGGTGCGGCTGGTATGGCAACAGCTGGTGGACACGGCTGCGCTGGTGATCGACCTGCGCTACAACCCTGGCAGCTACTCCACGGCCATCCCGCTGCTCTGCTCCTACTTCTTTGAGGCAGAGCCCCGCCAGCACCTGTATTCTGTCTTTGACAGGGCCACCTCAAAAGTCACGGAGGTGTGGACCTTGCCCCAGGTCGCCGGCCAGCGCTACGGCTCACACAAGGACCTCTACATCCTGATGAGCCACACCAGTGGCTCTGCGGCCGAGGCCTTTGCACACACCATGCAGGACCTGCAGCGGGCCACGGTCATTGGGGAGCCCACGGCCGGAGGCGCACTCTCTGTGGGCATCTACCAGGTGGGCAGCAGCCCCTTATATGCATCCATGCCCACCCAGATGGCCATGAGTGCCACCACAGGCAAGGCCTGGGACCTGGCTGGTGTGGAGCCCGACATCACTGTGCCCATGAGCGAAGCCCTTTCCATAGCCCAGGACATAGTGGCTCTGCGTGCCAAGGTGCCCACGGTGCTGCAGACGGCCGGGAAGCTGGTGGCTGATAACTATGCCTCTGCCGAGCTGGGGGCCAAGATGGCCACCAAACTGAGCGGTCTGCAGAGCCGCTACTCCAGGGTGACCTCAGAAGTGGCCCTAGCCGAGATCCTGGGGGCTGACCTGCAGATGCTCTCCGGAGACCCACACCTGAAGGCAGCCCATATCCCTGAGAATGCCAAGGACCGCATTCCTGGAATTGTGCCCATGCAGATCCCTTCCCCTGAAGTATTTGAAGAGCTGATCAAGTTTTCCTTCCACACTAACGTGCTTGAGGACAACATTGGCTACTTGAGGTTTGACATGTTTGGGGACGGTGAGCTGCTCACCCAGGTCTCCAGGCTGCTGGTGGAGCACATCTGGAAGAAGATCATGCACACGGATGCCATGATCATCGACATGAGGTTCAACATCGGTGGCCCCACATCCTCCATTCCCATCTTGTGCTCCTACTTCTTTGATGAAGGCCCTCCAGTTCTGCTGGACAAGATCTACAGCCGGCCTGATGACTCTGTCAGTGAACTCTGGACACACGCCCAGGTTGTAGGTGAACGCTATGGCTCCAAGAAGAGCATGGTCATTCTGACCAGCAGTGTGACGGCCGGCACCGCGGAGGAGTTCACCTATATCATGAAGAGGCTGGGCCGGGCCCTGGTCATTGGGGAGGTGACCAGTGGGGGCTGCCAGCCACCACAGACCTACCACGTGGATGACACCAACCTCTACCTCACTATCCCCACGGCCCGTTCTGTGGGGGCCTCGGATGGCAGCTCCTGGGAAGGGGTGGGGGTGACACCCCATGTGGTTGTCCCTGCAGAAGAGGCTCTCGCCAGGGCCAAGGAGATGCTCCAGCACAACCAGCTGAGGGTGAAGCGGAGCCCAGGCCTGCAGGACCACCTGTAGGGAAGGGCCCCATAGGCAGAGCCCCAGGGCAGACAGAACCTCTGGGACACACACCAAGGGCACTCCTGCAGGTGGCCCGGCCTGAGGTTCCCAGGAGCAGCAAAGGGGCCTGCTGAGCTCTGGTTAGGTTACAGCTGGAGGTGTGTATATATACACACACACACATGTATATACACATATATATGTGTATGTATATATATGTATATATATATGGCTTTCCAATAACCACCTAAATTTTAACAAAGGTTCCTTCTAAGTGGTAGAACTTGGGGTGGTATTTTTACCTTCCTTCTTCATACTTTGCTCTTTTTCTTAAATACTCATTAATGTGCATATATCATTATTTTCAGATGCAGCTATCATTATTCCAAAATACAAAATAAAGAAGATAAAATAAATTATATACCCGAGCCATTAAAAAAAAAAAAAAA